(19) United States
(12) Patent Application Publication
(10) Pub. No.: US 2025/0325664 A1
(43) Pub. Date: Oct. 23, 2025

(54) COMBINATION THERAPY WITH AN ANTI-COLONY STIMULATING FACTOR 1 RECEPTOR ANTIBODY AND A JAK INHIBITOR

(71) Applicants: Incyte Corporation, Wilmington, DE (US); Syndax Pharmaceuticals, Inc., Waltham, MA (US)

(72) Inventors: Peter Langmuir, Cherry Hill, NJ (US); Lena Ohannesian, Blue Bell, PA (US); Abhishek Dubey, Philadelphia, PA (US); Lea M. Burke, West Chester, PA (US); Rodica Morariu-Zamfir, Geneva (CH); Vedran Radojcic, Salt Lake City, UT (US)

(21) Appl. No.: 19/185,796

(22) Filed: Apr. 22, 2025

Related U.S. Application Data
(60) Provisional application No. 63/637,116, filed on Apr. 22, 2024.
(60) Provisional application No. 63/749,752, filed on Jan. 27, 2025.

(51) Int. Cl. A61K 39/395; A61K 31/519; A61P 37/06

(52) U.S. Cl. CPC A61K 39/3955; A61K 31/519; A61P 37/06

(57) ABSTRACT

The present disclosure describes a combination of an anti-colony stimulating factor 1 receptor antibody and a JAK inhibitor for the treatment of chronic graft-versus-host disease.

### CROSS-REFERENCE TO RELATED APPLICATIONS

This application claims priority to U.S. Provisional Application No. 63/637,116, filed on Apr. 22, 2024 and U.S. Provisional Application No. 63/749,752, filed on Jan. 27, 2025, the contents of which are hereby incorporated by reference.

### SEQUENCE LISTING

This application contains a Sequence Listing that has been submitted electronically as an XML file named “20443-0783001_SL_ST26.XML.” The XML file, created on Apr. 22, 2025, is 13,732 bytes in size. The material in the XML file is hereby incorporated by reference in its entirety.

### BACKGROUND

Chronic graft-versus-host disease (cGVHD) is a severe complication of allogeneic hematopoietic cell transplantation (HCT) that affects various organs leading to a reduced quality of life. cGVHD occurs in up to 50% of allogeneic HCT cases, where donor T- and B-cells derived from the graft recognize and attack host antigens (Socié et al., 2014, Blood, 124:374-84) and has increased during the last two decades due to increasing patient age and increasing use of unrelated and/or mismatched donors, reduced intensity conditioning regimens, and peripheral blood as source for stem cells (Arai et al., 2015, Biol Blood Marrow Transplant, 21:266-74). Due to an increased risk of non-relapse mortality, cGVHD remains the leading cause for late mortality following allo-HCT (Zeiser et al., 2018, Blood, 131:1399-405; Li et al., 2019, Br J Haematol, 184:323-36).

Treatment of cGVHD with systemic corticosteroids is often ineffective, with frequent incomplete responses and recurrences (Inamoto et al., 2014, Blood, 124:1363-71). About 50-60% of patients with cGVHD will require a second-line treatment within two years. Additionally, prolonged systemic corticosteroid treatment is associated with significant side effects, such as infections, myopathy, cataract, diabetes, hypertension, decline in bone mass, and avascular necrosis (Flowers et al., 2015, Blood, 125 (4): 606-15). Therefore, new therapeutic approaches are needed for treatment of cGVHD.

### SUMMARY

The disclosure features a method of treating chronic graft-versus-host disease (cGVHD) in a human subject in need thereof by administering to the human subject a therapeutically effective amount of a JAK inhibitor and an antibody that binds to colony stimulating factor 1 receptor (CSF-1R), wherein the antibody comprises a variable heavy (VH) domain comprising VH complementarity determining region (CDR) 1 (VH CDR1), VH CDR2, and VH CDR3, wherein:
the VH CDR1 comprises the amino acid sequence GFSLTTYGMGVG (SEQ ID NO:6);
the VH CDR2 comprises the amino acid sequence NIWWDDDKYYNPSLKN (SEQ ID NO:7); and
the VH CDR3 comprises the amino acid sequence IGPIKYPTAPYRYFDF (SEQ ID NO:8); and
wherein the antibody comprises a variable light (VL) domain comprising VL CDR1, VL CDR2, and VL CDR3, wherein:
the VL CDR1 comprises the amino acid sequence LASEDIYDNLA (SEQ ID NO:9);
the VL CDR2 comprises the amino acid sequence YASSLQD (SEQ ID NO:10); and
the VL CDR3 comprises the amino acid sequence LQDSEYPWT (SEQ ID NO:11).

In some embodiments, the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5).

In some embodiments, the antibody comprises a heavy chain and a light chain, wherein the heavy chain comprises the amino acid sequence set forth in SEQ ID NO:12 (or SEQ ID NO: 2) and the light chain comprises the amino acid sequence set forth in SEQ ID NO:3.

In some embodiments, the JAK inhibitor is a JAK1 inhibitor.

In some embodiments, the JAK inhibitor is a JAK2 inhibitor.

In some embodiments, the JAK inhibitor is a JAK 1/2 inhibitor.

In some embodiments, the JAK inhibitor is ruxolitinib.

In some embodiments, the JAK inhibitor is itacitinib.

In some embodiments, the JAK inhibitor is 4-[3-(cyanomethyl)-3-(3′,5′-dimethyl-1H,1′H-4,4′-bipyrazol-1-yl) azetidin-1-yl]-2,5-difluoro-N-[(1S)-2,2,2-trifluoro-1-methylethyl]benzamide or a pharmaceutically acceptable salt thereof, or ((2R,5S)-5-{2-[(1R)-1-hydroxyethyl]-1H-imidazo[4,5-d]thieno[3,2-b]pyridin-1-yl}tetrahydro-2H-pyran-2-yl) acetonitrile or a pharmaceutically acceptable salt thereof.

In some embodiments, the JAK inhibitor is a compound is a compound of Formula I:

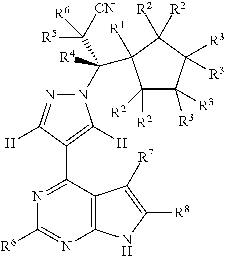

or a pharmaceutically acceptable salt thereof, wherein:
R¹ is selected from H and D;
each R² is independently selected from H and D, provided that each R² attached to a common carbon is the same;
each R³ is independently selected from H and D, provided that each R³ attached to a common carbon is the same;
R⁴ is selected from H and D;
each R⁵ is the same and is selected from H and D; and
R⁶, R⁷, and R⁸ are each independently selected from H and D; provided that when R¹ is H, each R² and each R³ are H, R⁴ is H, and each of R⁶, R⁷, and R⁸ is H, then each R⁵ is D.

In some embodiments, the cGVHD is newly diagnosed cGVHD.

In some embodiments, the cGVHD is moderate or severe cGVHD.

In some embodiments, the antibody is administered intravenously.

In some embodiments, the JAK inhibitor is administered orally.

In some embodiments, the JAK inhibitor is ruxolitinib and is administered orally at a dose of 1 mg to 50 mg.

In some embodiments, the JAK inhibitor is ruxolitinib and is administered orally twice per day at a dose of 1 mg to 50 mg.

In some embodiments, the JAK inhibitor is ruxolitinib and is administered orally at a dose of 10 mg.

In some embodiments, the JAK inhibitor is ruxolitinib and is administered orally twice per day at a dose of 10 mg.

In some embodiments, the antibody comprises a heavy chain and a light chain, wherein the heavy chain comprises the amino acid sequence set forth in SEQ ID NO: 12 (or SEQ ID NO: 2) and the light chain comprises the amino acid sequence set forth in SEQ ID NO:3, and the antibody is administered intravenously at a dose of 0.3 mg/kg.

In some embodiments, the antibody comprises a heavy chain and a light chain, wherein the heavy chain comprises the amino acid sequence set forth in SEQ ID NO: 12 (or SEQ ID NO: 2) and the light chain comprises the amino acid sequence set forth in SEQ ID NO:3, and the antibody is administered intravenously once every two weeks at a dose of 0.3 mg/kg.

In some embodiments, the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5), and the JAK inhibitor is ruxolitinib.

In some embodiments, the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5), the antibody is administered intravenously, and the JAK inhibitor is ruxolitinib and is administered orally.

In some embodiments, the antibody comprises a heavy chain and a light chain, wherein the heavy chain comprises the amino acid sequence set forth in SEQ ID NO:12 (or SEQ ID NO: 2) and the light chain comprises the amino acid sequence set forth in SEQ ID NO:3, and the JAK inhibitor is ruxolitinib.

In some embodiments, the antibody comprises a heavy chain and a light chain, wherein the heavy chain comprises the amino acid sequence set forth in SEQ ID NO: 12 (or SEQ ID NO: 2) and the light chain comprises the amino acid sequence set forth in SEQ ID NO:3, the antibody is administered intravenously, and the JAK inhibitor is ruxolitinib and is administered orally.

In some embodiments, the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5), the antibody is administered intravenously at a dose of 0.3 mg/kg, and the JAK inhibitor is ruxolitinib and is administered orally at a dose of 10 mg.

In some embodiments, the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5), the antibody is administered intravenously once every two weeks at a dose of 0.3 mg/kg, and the JAK inhibitor is ruxolitinib and is administered orally twice per day at a dose of 10 mg.

In some embodiments, the antibody comprises a heavy chain and a light chain, wherein the heavy chain comprises the amino acid sequence set forth in SEQ ID NO: 12 (or SEQ ID NO: 2) and the light chain comprises the amino acid sequence set forth in SEQ ID NO:3, the antibody is administered intravenously at a dose of 0.3 mg/kg, and the JAK inhibitor is ruxolitinib and is administered orally at a dose of 10 mg.

In some embodiments, the antibody comprises a heavy chain and a light chain, wherein the heavy chain comprises the amino acid sequence set forth in SEQ ID NO: 12 (or SEQ ID NO: 2) and the light chain comprises the amino acid sequence set forth in SEQ ID NO:3, the antibody is administered intravenously once every two weeks at a dose of 0.3 mg/kg, and the JAK inhibitor is ruxolitinib and is administered orally twice per day at a dose of 10 mg.

In some embodiments, the antibody comprises a heavy chain and a light chain, wherein the heavy chain comprises the amino acid sequence set forth in SEQ ID NO: 12 (or SEQ ID NO: 2) and the light chain comprises the amino acid sequence set forth in SEQ ID NO:3, and the antibody is administered intravenously at a dose of 0.6 mg/kg.

In some embodiments, the antibody comprises a heavy chain and a light chain, wherein the heavy chain comprises the amino acid sequence set forth in SEQ ID NO: 12 (or SEQ ID NO: 2) and the light chain comprises the amino acid sequence set forth in SEQ ID NO:3, and the antibody is administered intravenously once every four weeks at a dose of 0.6 mg/kg.

In some embodiments, the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5), the antibody is administered intravenously at a dose of 0.6 mg/kg, and the JAK inhibitor is ruxolitinib and is administered orally at a dose of 10 mg.

In some embodiments, the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5), the antibody is administered intravenously once every four weeks at a dose of 0.6 mg/kg, and the JAK inhibitor is ruxolitinib and is administered orally twice per day at a dose of 10 mg.

In some embodiments, the antibody comprises a heavy chain and a light chain, wherein the heavy chain comprises the amino acid sequence set forth in SEQ ID NO:12 (or SEQ ID NO: 2) and the light chain comprises the amino acid sequence set forth in SEQ ID NO:3, the antibody is administered intravenously at a dose of 0.6 mg/kg, and the JAK inhibitor is ruxolitinib and is administered orally at a dose of 10 mg.

In some embodiments, the antibody comprises a heavy chain and a light chain, wherein the heavy chain comprises the amino acid sequence set forth in SEQ ID NO:12 (or SEQ ID NO: 2) and the light chain comprises the amino acid sequence set forth in SEQ ID NO:3, the antibody is administered intravenously once every four weeks at a dose of 0.6 mg/kg, and the JAK inhibitor is ruxolitinib and is administered orally twice per day at a dose of 10 mg.

In some embodiments, the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5), and the JAK inhibitor is itacitinib.

In some embodiments, the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5), the antibody is administered intravenously, and the JAK inhibitor is itacitinib and is administered orally.

In some embodiments, the antibody comprises a heavy chain and a light chain, wherein the heavy chain comprises the amino acid sequence set forth in SEQ ID NO: 12 (or SEQ ID NO: 2) and the light chain comprises the amino acid sequence set forth in SEQ ID NO:3, and the JAK inhibitor is itacitinib.

In some embodiments, the antibody comprises a heavy chain and a light chain, wherein the heavy chain comprises the amino acid sequence set forth in SEQ ID NO: 12 (or SEQ ID NO: 2) and the light chain comprises the amino acid sequence set forth in SEQ ID NO:3, the antibody is administered intravenously, and the JAK inhibitor is itacitinib and is administered orally.

In some embodiments, the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5), and the JAK inhibitor is 4-[3-(cyanomethyl)-3-(3′,5′-dimethyl-1H,1′H-4,4′-bipyrazol-1-yl) azetidin-1-yl]-2,5-difluoro-N-[(1S)-2,2,2-trifluoro-1-methylethyl]benzamide or a pharmaceutically acceptable salt thereof.

In some embodiments, the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5), the antibody is administered intravenously, and the JAK inhibitor is 4-[3-(cyanomethyl)-3-(3′,5′-dimethyl-1H,1′H-4,4′-bipyrazol-1-yl) azetidin-1-yl]-2,5-difluoro-N-[(1S)-2,2,2-trifluoro-1-methylethyl]benzamide or a pharmaceutically acceptable salt thereof and is administered orally.

In some embodiments, the antibody comprises a heavy chain and a light chain, wherein the heavy chain comprises the amino acid sequence set forth in SEQ ID NO: 12 (or SEQ ID NO: 2) and the light chain comprises the amino acid sequence set forth in SEQ ID NO:3, and the JAK inhibitor is 4-[3-(cyanomethyl)-3-(3′,5′-dimethyl-1H, 1′H-4,4′-bipyrazol-1-yl) azetidin-1-yl]-2,5-difluoro-N-[(1S)-2,2,2-trifluoro-1-methylethyl]benzamide or a pharmaceutically acceptable salt thereof.

In some embodiments, the antibody comprises a heavy chain and a light chain, wherein the heavy chain comprises the amino acid sequence set forth in SEQ ID NO: 12 (or SEQ ID NO: 2) and the light chain comprises the amino acid sequence set forth in SEQ ID NO:3, the antibody is administered intravenously, and the JAK inhibitor is 4-[3-(cyanomethyl)-3-(3′,5′-dimethyl-1H,1′H-4,4′-bipyrazol-1-yl) azetidin-1-yl]-2,5-difluoro-N-[(1S)-2,2,2-trifluoro-1-methylethyl]benzamide or a pharmaceutically acceptable salt thereof and is administered orally.

In some embodiments, the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5), and the JAK inhibitor is ((2R,5S)-5-{2-[(1R)-1-hydroxyethyl]-1H-imidazo[4,5-d]thieno[3,2-b]pyridin-1-yl}tetrahydro-2H-pyran-2-yl) acetonitrile or a pharmaceutically acceptable salt thereof.

In some embodiments, the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5), the antibody is administered intravenously, and the JAK inhibitor is ((2R,5S)-5-{2-[(1R)-1-hydroxyethyl]-1H-imidazo[4,5-d]thieno[3,2-b]pyridin-1-yl}tetrahydro-2H-pyran-2-yl) acetonitrile or a pharmaceutically acceptable salt thereof and is administered orally.

In some embodiments, the antibody comprises a heavy chain and a light chain, wherein the heavy chain comprises the amino acid sequence set forth in SEQ ID NO: 12 (or SEQ ID NO: 2) and the light chain comprises the amino acid sequence set forth in SEQ ID NO:3, and the JAK inhibitor is ((2R,5S)-5-{2-[(1R)-1-hydroxyethyl]-1H-imidazo[4,5-d]thieno[3,2-b]pyridin-1-yl}tetrahydro-2H-pyran-2-yl) acetonitrile or a pharmaceutically acceptable salt thereof.

In some embodiments, the antibody comprises a heavy chain and a light chain, wherein the heavy chain comprises the amino acid sequence set forth in SEQ ID NO: 12 (or SEQ ID NO: 2) and the light chain comprises the amino acid sequence set forth in SEQ ID NO:3, the antibody is administered intravenously, and the JAK inhibitor is ((2R,5S)-5-{2-[(1R)-1-hydroxyethyl]-1H-imidazo[4,5-d]thieno[3,2-b]pyridin-1-yl}tetrahydro-2H-pyran-2-yl) acetonitrile or a pharmaceutically acceptable salt thereof and is administered orally.

In some embodiments, the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5), and the JAK inhibitor is a compound of Formula I:

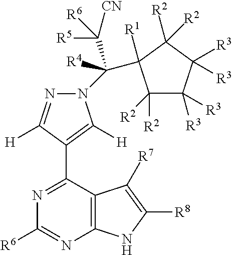

or a pharmaceutically acceptable salt thereof, wherein:
R¹ is selected from H and D;
each R² is independently selected from H and D, provided that each R² attached to a common carbon is the same;
each R³ is independently selected from H and D, provided that each R³ attached to a common carbon is the same;
R⁴ is selected from H and D;
each R⁵ is the same and is selected from H and D; and
R⁶, R⁷, and R⁸ are each independently selected from H and D; provided that when R¹ is H, each R² and each R³ are H, R⁴ is H, and each of R⁶, R⁷, and R⁸ is H, then each R⁵ is D.

In some embodiments, the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5), the antibody is administered intravenously, and the JAK inhibitor is a compound of Formula I:

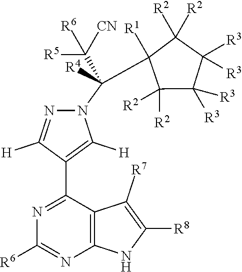

or a pharmaceutically acceptable salt thereof, wherein:
R¹ is selected from H and D;
each R² is independently selected from H and D, provided that each R² attached to a common carbon is the same;
each R³ is independently selected from H and D, provided that each R³ attached to a common carbon is the same;
R⁴ is selected from H and D;
each R⁵ is the same and is selected from H and D; and
R⁶, R⁷, and R⁸ are each independently selected from H and D; provided that when R¹ is H, each R² and each R³ are H, R⁴ is H, and each of R⁶, R⁷, and R⁸ is H, then each R⁵ is D and is administered orally.

In some embodiments, the antibody comprises a heavy chain and a light chain, wherein the heavy chain comprises the amino acid sequence set forth in SEQ ID NO: 12 (or SEQ ID NO: 2) and the light chain comprises the amino acid sequence set forth in SEQ ID NO:3, and the JAK inhibitor is a compound of Formula I:

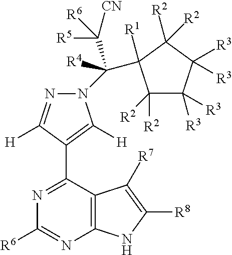

or a pharmaceutically acceptable salt thereof, wherein:
R¹ is selected from H and D;
each R² is independently selected from H and D, provided that each R² attached to a common carbon is the same;
each R³ is independently selected from H and D, provided that each R³ attached to a common carbon is the same;
R⁴ is selected from H and D;
each R⁵ is the same and is selected from H and D; and
R⁶, R⁷, and R⁸ are each independently selected from H and D; provided that when R¹ is H, each R² and each R³ are H, R⁴ is H, and each of R⁶, R⁷, and R⁸ is H, then each R⁵ is D.

In some embodiments, the antibody comprises a heavy chain and a light chain, wherein the heavy chain comprises the amino acid sequence set forth in SEQ ID NO:12 (or SEQ ID NO: 2) and the light chain comprises the amino acid sequence set forth in SEQ ID NO:3, the antibody is administered intravenously, and the JAK inhibitor is a compound of Formula I:

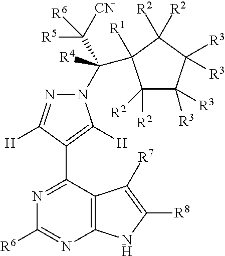

or a pharmaceutically acceptable salt thereof, wherein:
R¹ is selected from H and D;
each R² is independently selected from H and D, provided that each R² attached to a common carbon is the same;
each R³ is independently selected from H and D, provided that each R³ attached to a common carbon is the same;
R⁴ is selected from H and D;
each R⁵ is the same and is selected from H and D; and
R⁶, R⁷, and R⁸ are each independently selected from H and D; provided that when R¹ is H, each R² and each R³ are H, R⁴ is H, and each of R⁶, R⁷, and R⁸ is H, then each R⁵ is D and is administered orally.

Other features and advantages of the invention will be apparent from the following detailed description, and from the claims.

### DETAILED DESCRIPTION

The combination of an anti-colony stimulating factor 1 receptor antibody (anti-CSF-1R antibody) and a JAK inhibitor can be used to treat chronic graft-versus-host disease.

### Anti-Colony Stimulating Factor 1 Receptor Antibodies

Axatilimab (also known as SNDX-6352) is a humanized IgG4 monoclonal antibody that binds to colony-stimulating factor 1 receptor (CSF-1R) and inhibits its function. Axatilimab is described in U.S. Pat. No. 9,908,939, which is incorporated by reference in its entirety. CSF-1R is a receptor for the cytokine CSF-1, which is responsible for the production, differentiation, and function of macrophages.

The amino acid sequence of the human CSF-1R protein is:

(SEQ ID NO: 1)
MGPGVLLLLLVATAWHGQGIPVIEPSVPELVVKPGATVTLRCVGN
GSVEWDGPPSPHWTLYSDGSSSILSTNNATFQNTGTYRCTEPGDP
LGGSAAIHLYVKDPARPWNVLAQEVVVFEDQDALLPCLLTDPVLE
AGVSLVRVRGRPLMRHTNYSFSPWHGFTIHRAKFIQSQDYQCSAL
MGGRKVMSISIRLKVQKVIPGPPALTLVPAELVRIRGEAAQIVCS
ASSVDVNFDVFLQHNNTKLAIPQQSDFHNNRYQKVLTLNLDQVDF
QHAGNYSCVASNVQGKHSTSMFFRVVESAYLNLSSEQNLIQEVTV
GEGLNLKVMVEAYPGLQGFNWTYLGPFSDHQPEPKLANATTKDTY
RHTFTLSLPRLKPSEAGRYSFLARNPGGWRALTFELTLRYPPEVS
VIWTFINGSGTLLCAASGYPQPNVTWLQCSGHTDRCDEAQVLQVW
DDPYPEVLSQEPFHKVTVQSLLTVETLEHNQTYECRAHNSVGSGS
WAFIPISAGAHTHPPDEFLFTPVVVACMSIMALLLLLLLLLLYKY
KQKPKYQVRWKIIESYEGNSYTFIDPTQLPYNEKWEFPRNNLQFG
KTLGAGAFGKVVEATAFGLGKEDAVLKVAVKMLKSTAHADEKEAL
MSELKIMSHLGQHENIVNLLGACTHGGPVLVITEYCCYGDLLNFL
RRKAEAMLGPSLSPGQDPEGGVDYKNIHLEKKYVRRDSGFSSQGV
DTYVEMRPVSTSSNDSFSEQDLDKEDGRPLELRDLLHFSSQVAQG
MAFLASKNCIHRDVAARNVLLTNGHVAKIGDFGLARDIMNDSNYI
VKGNARLPVKWMAPESIFDCVYTVQSDVWSYGILLWEIFSLGLNP
YPGILVNSKFYKLVKDGYQMAQPAFAPKNIYSIMQACWALEPTHR
PTFQQICSFLQEQAQEDRRERDYTNLPSSSRSGGSGSSSSELEEE
SSSEHLTCCEQGDIAQPLLQPNNYQFC.

The amino acid sequences of axatilimab heavy and light chains are shown below. Complementarity-determining regions (CDRs) 1, 2, and 3 of the variable heavy (VH) domain and the variable light (VL) domain are shown in that order from N-terminus to the C-terminus of the mature VL and VH sequences and are both underlined and boldened. Variable regions are underlined. An antibody consisting of the heavy chain (SEQ ID NO:2) and the light chain (SEQ ID NO: 3) listed below is termed axatilimab.

### Axatilimab Heavy Chain:

(SEQ ID NO: 2)
EVTLKESGPALVKPTQTLTLTCTFSGFSLTTYGMGVGWIRQPPGK
ALEWLANIWWDDDKYYNPSLKNRLTISKDTSKNQVVLTMTNMDPV
DTATYYCARIGPIKYPTAPYRYFDFWGQGTMVTVSSASTKGPSVF
PLAPCSRSTSESTAALGCLVKDYFPEPVTVSWNSGALTSGVHTFP
AVLQSSGLYSLSSVVTVPSSSLGTKTYTCNVDHKPSNTKVDKRVE
SKYGPPCPPCPAPEFLGGPSVFLFPPKPKDTLMISRTPEVTCVVV
DVSQEDPEVQFNWYVDGVEVHNAKTKPREEQFNSTYRVVSVLTVL
HQDWLNGKEYKCKVSNKGLPSSIEKTISKAKGQPREPQVYTLPPS
QEEMTKNQVSLTCLVKGFYPSDIAVEWESNGQPENNYKTTPPVLD
SDGSFFLYSRLTVDKSRWQEGNVFSCSVMHEALHNHYTQKSLSLS
LGK.

### Axatilimab Light Chain:

(SEQ ID NO: 3)
DIQMTQSPSSLSASVGDRVTITCLASEDIYDNLAWYQQKPGKAPK
LLIYYASSLQDGVPSRFSGSGSGTDYTLTISSLQPEDFATYYCLQ
DSEYPWTFGGGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC.

The variable heavy (VH) domain of axatilimab has the following amino acid sequence:

(SEQ ID NO: 4)
EVTLKESGPALVKPTQTLTLTCTFSGFSLTTYGMGVGWIRQPPGK
ALEWLANIWWDDDKYYNPSLKNRLTISKDTSKNQVVLTMTNMDPV
DTATYYCARIGPIKYPTAPYRYFDFWGQGTMVTVS

The variable light (VL) domain of axatilimab has the following amino acid sequence:

(SEQ ID NO: 5)
DIQMTQSPSSLSASVGDRVTITCLASEDIYDNLAWYQQKPGKAPK
LLIYYASSLQDGVPSRFSGSGSGTDYTLTISSLQPEDFATYYCLQ
DSEYPWTFGGGTKVEIK

The amino acid sequences of the VH CDRs of axatilimab are listed below:

VH CDR1:
(SEQ ID NO: 6)
GFSLTTYGMGVG;
VH CDR2:
(SEQ ID NO: 7)
NIWWDDDKYYNPSLKN;
and
VH CDR3:
(SEQ ID NO: 8)
IGPIKYPTAPYRYFDF.

The amino acid sequences of the VL CDRs of axatilimab are listed below:

VL CDR1:
(SEQ ID NO: 9)
LASEDIYDNLA;
VL CDR2:
(SEQ ID NO: 10)
YASSLQD;
and
VL CDR3:
(SEQ ID NO: 11)
LQDSEYPWT.

In certain embodiments, the anti-CSF-1R antibody includes a human heavy chain and light chain constant region. In certain embodiments, the heavy chain constant region comprises a CH1 domain and a hinge region. In some embodiments, the heavy chain constant region comprises a CH2 domain. In some embodiments, the heavy chain constant region comprises a CH3 domain. In some embodiments, the heavy chain constant region comprises CH1, CH2 and CH3 domains. If the heavy chain constant region includes substitutions, such substitutions modify the properties of the antibody (e.g., increase or decrease one or more of: Fc receptor binding, antibody glycosylation, the number of cysteine residues, effector cell function, or complement function). In certain embodiments, the antibody is an IgG antibody. In specific embodiments, the antibody is selected from the group consisting of IgG1, IgG2, IgG3, and IgG4.

In some embodiments, an anti-CSF-1R antibody is provided wherein a C-terminal residue of an antibody sequence described herein is cleaved, for example, the C-terminal residue of a heavy chain sequence, for example, a terminal lysine. Generally, the cleavage results from post-translation modifications of the expressed antibody. For example, an anti-CSF-1R antibody can include a heavy chain comprising the amino acid sequence set forth in SEQ ID NO:12 (below) and a light chain comprising the amino acid sequence set forth in SEQ ID NO:3.

(SEQ ID NO: 12)
EVTLKESGPALVKPTQTLTLTCTFSGFSLTTYGMGVGWIRQPPGK
ALEWLANIWWDDDKYYNPSLKNRLTISKDTSKNQVVLTMTNMDPV
DTATYYCARIGPIKYPTAPYRYFDFWGQGTMVTVSSASTKGPSVF
PLAPCSRSTSESTAALGCLVKDYFPEPVTVSWNSGALTSGVHTFP
AVLQSSGLYSLSSVVTVPSSSLGTKTYTCNVDHKPSNTKVDKRVE
SKYGPPCPPCPAPEFLGGPSVFLFPPKPKDTLMISRTPEVTCVVV
DVSQEDPEVQFNWYVDGVEVHNAKTKPREEQFNSTYRVVSVLTVL
HQDWLNGKEYKCKVSNKGLPSSIEKTISKAKGQPREPQVYTLPPS
QEEMTKNQVSLTCLVKGFYPSDIAVEWESNGQPENNYKTTPPVLD
SDGSFFLYSRLTVDKSRWQEGNVFSCSVMHEALHNHYTQKSLSLS
LG

Antibodies, such as axatilimab, can be made, for example, by preparing and expressing synthetic genes that encode the recited amino acid sequences or by mutating human germline genes to provide a gene that encodes the recited amino acid sequences. Moreover, this antibody and other anti-CSF-1R antibodies can be obtained, e.g., using one or more of the following methods.

Humanized antibodies can be generated by replacing sequences of the Fv variable region that are not directly involved in antigen binding with equivalent sequences from human Fv variable regions. General methods for generating humanized antibodies are provided by Morrison, S. L., Science, 229:1202-1207 (1985), by Oi et al., BioTechniques, 4:214 (1986), and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target, as described above, from germline immunoglobulin genes, or from synthetic constructs. The recombinant DNA encoding the humanized antibody can then be cloned into an appropriate expression vector.

Human germline sequences, for example, are disclosed in Tomlinson, I. A. et al., J. Mol. Biol., 227:776-798 (1992); Cook, G. P. et al., Immunol. Today, 16:237-242 (1995); Chothia, D. et al., J. Mol. Bio. 227:799-817 (1992); and Tomlinson et al., EMBO J., 14:4628-4638 (1995). The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

Other methods for humanizing antibodies can also be used. For example, other methods can account for the three dimensional structure of the antibody, framework positions that are in three dimensional proximity to binding determinants, and immunogenic peptide sequences. See, e.g., WO 90/07861; U.S. Pat. Nos. 5,693,762; 5,693,761; 5,585,089; 5,530,101; and U.S. Pat. No. 6,407,213; Tempest et al. (1991) Biotechnology 9:266-271. Still another method is termed “humaneering” and is described, for example, in U.S. 2005-008625.

The antibody can include a human Fc region, e.g., a wild-type Fc region or an Fc region that includes one or more alterations. Antibodies may also have mutations that stabilize the disulfide bond between the two heavy chains of an immunoglobulin, such as mutations in the hinge region of IgG4, as disclosed in the art (e.g., Angal et al. (1993) Mol. Immunol. 30:105-08). See also, e.g., U.S. 2005-0037000.

The anti-CSF-1R antibodies can be in the form of full length antibodies, or in the form of low molecular weight forms (e.g., biologically active antibody fragments or minibodies) of the anti-CSF-1R antibodies, e.g., Fab, Fab′, F(ab′)₂, Fv, Fd, dAb, scFv, and sc (Fv)₂. Other anti-CSF-1R antibodies encompassed by this disclosure include single domain antibody (sdAb) containing a single variable chain such as, VH or VL, or a biologically active fragment thereof. See, e.g., Moller et al., J. Biol. Chem., 285 (49): 38348-38361 (2010); Harmsen et al., Appl. Microbiol. Biotechnol., 77 (1): 13-22 (2007); U.S. 2005/0079574 and Davies et al. (1996) Protein Eng., 9 (6): 531-7. Like a whole antibody, a sdAb is able to bind selectively to a specific antigen. With a molecular weight of only 12-15 kDa, sdAbs are much smaller than common antibodies and even smaller than Fab fragments and single-chain variable fragments.

Provided herein are compositions comprising a mixture of an anti-CSF-1R antibody and one or more acidic variants thereof, e.g., wherein the amount of acidic variant(s) in the composition is less than about 80%, 70%, 60%, 60%, 50%, 40%, 30%, 30%, 20%, 10%, 5% or 1%. Also provided are compositions comprising an anti-CSF-1R antibody comprising at least one deamidation site, wherein the pH of the composition is from about 5.0 to about 6.5, such that, e.g., at least about 90% of the anti-CSF-1R antibodies are not deamidated (i.e., less than about 10% of the antibodies are deamidated). In certain embodiments, less than about 5%, 3%, 2% or 1% of the antibodies are deamidated. The pH may be from 5.0 to 6.0, such as 5.5 or 6.0. In certain embodiments, the pH of the composition is 5.0, 5.1, 5.2, 5.3, 5.4, 5.5, 5.6, 5.7, 5.8, 5.9, 6.0, 6.1, 6.2, 6.3, 6.4 or 6.5.

An “acidic variant” is a variant of a polypeptide of interest which is more acidic (e.g., as determined by cation exchange chromatography) than the polypeptide of interest. An example of an acidic variant is a deamidated variant.

A “deamidated” variant of a polypeptide molecule is a polypeptide wherein one or more asparagine residue(s) of the original polypeptide have been converted to aspartate, i.e., the neutral amide side chain has been converted to a residue with an overall acidic character.

The term “mixture” as used herein in reference to a composition comprising an anti-CSF-1R antibody means the presence of both the desired anti-CSF-1R antibody and one or more acidic variants thereof. The acidic variants may comprise predominantly deamidated anti-CSF-1R antibody, with minor amounts of other acidic variant(s).

In certain embodiments, the binding affinity (K_{D}), on-rate (K_{D} on) and/or off-rate (K_{D} off) of the antibody that was mutated to eliminate deamidation is similar to that of the wild-type antibody, e.g., having a difference of less than about 5 fold, 2 fold, 1 fold (100%), 50%, 30%, 20%, 10%, 5%, 3%, 2% or 1%.

### Bispecific Antibodies

In certain embodiments, an anti-CSF-1R antibody described herein is present in a bispecific antibody. Bispecific antibodies are antibodies that have binding specificities for at least two different epitopes. Exemplary bispecific antibodies may bind to two different epitopes of the CSF-1R protein. Other such antibodies may combine a CSF-1R binding site with a binding site for another protein. Bispecific antibodies can be prepared as full length antibodies or low molecular weight forms thereof (e.g., F(ab′)₂ bispecific antibodies, sc(Fv)₂ bispecific antibodies, diabody bispecific antibodies).

Traditional production of full length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the CH3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

### Multivalent Antibodies

In certain embodiments, an anti-CSF-1R antibody thereof described herein is present in a multivalent antibody. A multivalent antibody may be internalized (and/or catabolized) faster than a bivalent antibody by a cell expressing an antigen to which the antibodies bind. The antibodies describe herein can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. An exemplary dimerization domain comprises (or consists of) an Fc region or a hinge region. A multivalent antibody can comprise (or consist of) three to about eight (e.g., four) antigen binding sites. The multivalent antibody optionally comprises at least one polypeptide chain (e.g., at least two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is a polypeptide chain of an Fc region, X1 and X2 represent an amino acid or peptide spacer, and n is 0 or 1.

### Conjugated Antibodies

The antibodies disclosed herein may be conjugated antibodies which are bound to various molecules including macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), hyaluronic acid, radioactive materials (e.g., ⁹⁰Y, ¹³¹I) fluorescent substances, luminescent substances, haptens, enzymes, metal chelates, drugs, and toxins (e.g., calicheamicin, Pseudomonas exotoxin A, ricin (e.g., deglycosylated ricin A chain)).

In one embodiment, to improve the cytotoxic actions of anti-CSF-1R antibodies and consequently their therapeutic effectiveness, the antibodies are conjugated with highly toxic substances, including radioisotopes and cytotoxic agents. These conjugates can deliver a toxic load selectively to the target site (i.e., cells expressing the antigen recognized by the antibody) while cells that are not recognized by the antibody are spared. In order to minimize toxicity, conjugates are generally engineered based on molecules with a short serum half-life (thus, the use of murine sequences, and IgG3 or IgG4 isotypes).

In certain embodiments, an anti-CSF-1R antibody is modified with a moiety that improves its stabilization and/or retention in circulation, e.g., in blood, serum, or other tissues, e.g., by at least 1.5, 2, 5, 10, or 50 fold. For example, the anti-CSF-1R antibody can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500 Daltons) can be used. For example, the anti-CSF-1R antibody can be conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides.

The above-described conjugated antibodies can be prepared by performing chemical modifications on the antibodies or the lower molecular weight forms thereof described herein. Methods for modifying antibodies are well known in the art (e.g., U.S. Pat. Nos. 5,057,313 and 5,156,840).

### Methods of Producing Antibodies

Antibodies may be produced in, for example, bacterial or eukaryotic cells. Some antibodies, e.g., Fab's, can be produced in bacterial cells, e.g., E. coli cells. Antibodies can also be produced in eukaryotic cells such as transformed cell lines (e.g., CHO, 293E, COS). In addition, antibodies (e.g., scFv's) can be expressed in a yeast cell such as Pichia (see, e.g., Powers et al., J Immunol Methods. 251:123-35 (2001)), Hansenula, or Saccharomyces. To produce the antibody of interest, a polynucleotide encoding the antibody is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DH5α, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, and NIH3T3 cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, antibodies are produced in mammalian cells. Exemplary mammalian host cells for expressing an antibody include Chinese Hamster Ovary (CHO cells) (including dhfr⁻ CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), human embryonic kidney 293 cells (e.g., 293, 293E, 293T), COS cells, NIH3T3 cells, lymphocytic cell lines, e.g., NS0 myeloma cells and SP2 cells, and a cell from a transgenic animal, e.g., a transgenic mammal. For example, the cell is a mammary epithelial cell.

In an exemplary system for antibody expression, a recombinant expression vector encoding both the antibody heavy chain and the antibody light chain of an anti-CSF-1R antibody (e.g., axatilimab) is introduced into dhfr CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

The antibodies of the present disclosure can be isolated from inside or outside (such as medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

### Antibody Pharmaceutical Compositions and Administration

An anti-CSF-1R antibody described herein can be formulated as a pharmaceutical composition for administration to a human subject, e.g., to treat a disorder described herein. Typically, a pharmaceutical composition includes a pharmaceutically acceptable carrier. As used herein, pharmaceutically acceptable carrier includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see e.g., Berge, S. M., et al. (1977) J. Pharm. Sci. 66:1-19).

Pharmaceutical formulation is a well-established art, and is further described, e.g., in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7th Ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X).

The anti-CSF-1R antibody can be administered to a human subject, e.g., a human subject in need thereof, for example, by a variety of methods. For many applications, the route of administration is one of: intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection. In some cases, administration can be oral.

The route and/or mode of administration of the antibody can also be tailored for the individual case, e.g., by monitoring the human subject, e.g., using tomographic imaging, e.g., to visualize a tumor.

The antibody can be administered as a fixed dose, or in a mg/kg subject weight dose (as used herein, “mg/kg” refers to mg of an antibody administered per kg of body weight of the treated subject). The dose can also be chosen to reduce or avoid production of antibodies against the anti-CSF-1R antibody. Dosage regimens are adjusted to provide the desired response, e.g., a therapeutic response or a combinatorial therapeutic effect. Generally, doses of the anti-CSF-1R antibody can be used in order to provide a human subject with the agent in bioavailable quantities. For example, doses in the range of about 0.1 mg/kg to about 30 mg/kg can be administered. In specific embodiments, a human subject is administered the antibody at a dose of about 0.3 mg/kg or about 0.6 mg/kg. As used herein, “about” when referring to a measurable value such as an amount, a dosage, a temporal duration, and the like, is meant to encompass variations of ±10%. For example, with respect to doses or dosages, the term “about” denotes a range that is ±10% of a recited dose, such that, for example, a dose of about 0.3 mg/kg will be between 0.27 mg/kg and 0.33 mg/kg subject weight. Exemplary doses included within “about 0.3 mg/kg” are 0.27 mg/kg, 0.28 mg/kg, 0.29 mg/kg, 0.3 mg/kg, 0.31 mg/kg, 0.32 mg/kg, and 0.33 mg/kg. Exemplary doses included within “about 0.6 mg/kg” are 0.54 mg/kg, 0.55 mg/kg, 0.56 mg/kg, 0.57 mg/kg, 0.58 mg/kg, 0.59 mg/kg, 0.6 mg/kg, 0.61 mg/kg, 0.62 mg/kg, 0.63 mg/kg, 0.64 mg/kg, 0.65 mg/kg, and 0.66 mg/kg.

Dosage unit form or “fixed dose” or “flat dose” as used herein refers to physically discrete units suited as unitary dosages for the human subjects to be treated; each unit contains a predetermined quantity of active compound calculated to produce the desired therapeutic effect in association with the required pharmaceutical carrier and optionally in association with the other agent. Single or multiple dosages may be given. Alternatively, or in addition, the antibody may be administered via continuous infusion.

An anti-CSF-1R antibody dose can be administered, e.g., at a periodic interval over a period of time (a course of treatment) sufficient to encompass at least 2 doses, 3 doses, 5 doses, 10 doses, or more, e.g., once or twice daily, or about one to four times per week (e.g., at least twice per week), or weekly, biweekly (every two weeks), every three weeks, every four weeks, monthly, e.g., for between about 1 to 12 weeks. Factors that may influence the dosage and timing required to effectively treat a human subject, include, e.g., the severity of the disease or disorder, formulation, route of delivery, previous treatments, the general health and/or age of the human subject, and other diseases present. Moreover, treatment of a human subject with a therapeutically effective amount of a compound can include a single treatment or, preferably, can include a series of treatments.

An exemplary weight-based dosing regimen comprises intravenous administration of an anti-CSF-1R antibody (e.g., axatilimab) at a dosage of about 0.3 mg/kg once every two weeks.

A further exemplary weight-based dosing regimen comprises intravenous administration of an anti-CSF-1R antibody (e.g., axatilimab) at a dosage of about 0.6 mg/kg once every four weeks.

A pharmaceutical composition may include a therapeutically effective amount of an anti-CSF-1R antibody described herein. The term “therapeutically effective amount” of an agent is that amount sufficient to effect beneficial or desired results, for example, clinical results, and, as such, a “therapeutically effective amount” depends upon the context in which it is being applied. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects.

### JAK Inhibitors

In some embodiments, the JAK inhibitor is a compound that inhibits JAK1, JAK2, JAK3, and/or TYK2. In some embodiments, the JAK inhibitor is selective for JAK1 and JAK2 over JAK3 and TYK2. In some embodiments, the JAK inhibitor is selective for JAK1 over JAK2, JAK3, and TYK2. For example, some of the compounds described herein, or a pharmaceutically acceptable salt thereof, preferentially inhibit JAK1 over one or more of JAK2, JAK3, and TYK2. In some embodiments, the compounds or salts inhibit JAK1 preferentially over JAK2 (e.g., have a JAK2/JAK1 IC₅₀ ratio >1). In some embodiments, the compounds or salts are about 10-fold more selective for JAK1 over JAK2. In some embodiments, the compounds or salts are about 3-fold, about 5-fold, about 10-fold, about 15-fold, or about 20-fold more selective for JAK1 over JAK2 as calculated by measuring IC₅₀ at 1 mM ATP.

In some embodiments, the JAK inhibitor is 3-cyclopentyl-3-[4-(7H-pyrrolo[2,3-d]pyrimidin-4-yl)-1H-pyrazol-1-yl]propanenitrile.

In some embodiments, the JAK inhibitor is (3R)-3-cyclopentyl-3-[4-(7H-pyrrolo[2,3-d]pyrimidin-4-yl)-1H-pyrazol-1-yl]propanenitrile (ruxolitinib; also known as INCB018424). 3-Cyclopentyl-3-[4-(7H-pyrrolo[2,3-d]pyrimidin-4-yl)-1H-pyrazol-1-yl]propanenitrile and ruxolitinib can be made by the procedure described in U.S. Pat. No. 7,598,257 (Example 67), filed Dec. 12, 2006, which is incorporated herein by reference in its entirety.

In some embodiments, the JAK inhibitor is (3R)-3-cyclopentyl-3-[4-(7H-pyrrolo[2,3-d]pyrimidin-4-yl)-1H-pyrazol-1-yl]propanenitrile phosphoric acid salt.

In some embodiments, the JAK inhibitor is baricitinib, tofacitinib, oclacitinib, filgotinib, gandotinib, lestaurtinib, momelotinib, bacritinib, PF-04965842, upadacitinib, peficitinib, fedratinib, cucurbitacin I, ATI-501 (Aclaris), ATI-502 (Aclaris), JTE052 (Leo Pharma and Japan Tobacco), or CHZ868.

In some embodiments, the JAK inhibitor can be an isotopically-labeled compound, or a pharmaceutically acceptable salt thereof. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to 2H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵O and ¹³¹I. For example, one or more hydrogen atoms in a compound of the present disclosure can be replaced by deuterium atoms (e.g., one or more hydrogen atoms of a C₁₋₆ alkyl group of Formula I can be optionally substituted with deuterium atoms, such as —CD₃ being substituted for —CH₃).

One or more constituent atoms of the compounds described herein can be replaced or substituted with isotopes of the atoms in natural or non-natural abundance. In some embodiments, the compound includes at least one deuterium atom. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

Accordingly, in some embodiments, the JAK inhibitor is a compound, wherein one or more hydrogen atoms in the compound are replaced by deuterium atoms, or a pharmaceutically acceptable salt thereof.

In some embodiments, the JAK inhibitor is ruxolitinib, wherein one or more hydrogen atoms are replaced by deuterium atoms, or a pharmaceutically acceptable salt thereof. In some embodiments, the JAK inhibitor is any of the compounds disclosed in U.S. Pat. No. 9,249,149 (which is incorporated herein by reference in its entirety), or a pharmaceutically acceptable salt thereof. In some embodiments, the JAK inhibitor is CTP-543, or a pharmaceutically acceptable salt thereof. In some embodiments, the JAK inhibitor is a compound of Formula I:

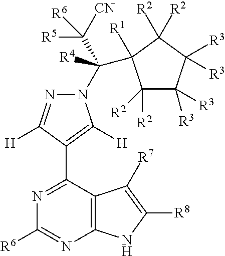

or a pharmaceutically acceptable salt thereof, wherein:
R¹ is selected from H and D;
each R² is independently selected from H and D, provided that each R² attached to a common carbon is the same;
each R³ is independently selected from H and D, provided that each R³ attached to a common carbon is the same;
R⁴ is selected from H and D;
each R⁵ is the same and is selected from H and D; and
R⁶, R⁷, and R⁸ are each independently selected from H and D; provided that when R¹ is H, each R² and each R³ are H, R⁴ is H, and each of R⁶, R⁷, and R⁸ is H, then each R⁵ is D.

In some embodiments, the JAK inhibitor is a compound of Formula I selected from the following compounds 100-130 in the table below (wherein R⁶, R⁷, and R⁸ are each H), or a pharmaceutically acceptable salt thereof. In some embodiments, the JAK inhibitor is a compound of Formula I selected from the following compounds 200-231 in the table below (wherein R⁶, R⁷, and R⁸ are each D), or a pharmaceutically acceptable salt thereof.

| Compound | R¹ | Each R² | Each R³ | R⁴ | Each R⁵ |
|---|---|---|---|---|---|
| 100 | H | H | H | D | H |
| 101 | H | H | H | H | D |
| 102 | H | H | H | D | D |
| 103 | H | H | D | H | H |
| 104 | H | H | D | D | H |
| 105 | H | H | D | H | D |
| 106 | H | H | D | D | D |
| 107 | H | D | H | H | H |
| 108 | H | D | H | D | H |
| 109 | H | D | H | H | D |
| 110 | H | D | H | D | D |
| 111 | H | D | D | H | H |
| 112 | H | D | D | D | H |
| 113 | H | D | D | H | D |
| 114 | H | D | D | D | D |
| 115 | D | H | H | H | H |
| 116 | D | H | H | D | H |
| 117 | D | H | H | H | D |
| 118 | D | H | H | D | D |
| 119 | D | H | D | H | H |
| 120 | D | H | D | D | H |
| 121 | D | H | D | H | D |
| 122 | D | H | D | D | D |
| 123 | D | D | H | H | H |
| 124 | D | D | H | D | H |
| 125 | D | D | H | H | D |
| 126 | D | D | H | D | D |
| 127 | D | D | D | H | H |
| 128 | D | D | D | D | H |
| 129 | D | D | D | H | D |
| 130 | D | D | D | D | D |
| 200 | H | H | H | D | H |
| 201 | H | H | H | H | D |
| 202 | H | H | H | D | D |
| 203 | H | H | D | H | H |
| 204 | H | H | D | D | H |
| 205 | H | H | D | H | D |
| 206 | H | H | D | D | D |
| 207 | H | D | H | H | H |
| 208 | H | D | H | D | H |
| 209 | H | D | H | H | D |
| 210 | H | D | H | D | D |
| 211 | H | D | D | H | H |
| 212 | H | D | D | D | H |
| 213 | H | D | D | H | D |
| 214 | H | D | D | D | D |
| 215 | D | H | H | H | H |
| 216 | D | H | H | D | H |
| 217 | D | H | H | H | D |
| 218 | D | H | H | D | D |
| 219 | D | H | D | H | H |
| 220 | D | H | D | D | H |
| 221 | D | H | D | H | D |
| 222 | D | H | D | D | D |
| 223 | D | D | H | H | H |
| 224 | D | D | H | D | H |
| 225 | D | D | H | H | D |
| 226 | D | D | H | D | D |
| 227 | D | D | D | H | H |
| 228 | D | D | D | D | H |
| 229 | D | D | D | H | D |
| 230 | D | D | D | D | D |
| 231 | H | H | H | H | H |

In some embodiments, the JAK inhibitor is baricitinib, wherein one or more hydrogen atoms are replaced by deuterium atoms, or a pharmaceutically acceptable salt thereof. In some embodiments, the JAK inhibitor is any of the compounds in U.S. Pat. No. 9,540,367 (which is incorporated herein by reference in its entirety), or a pharmaceutically acceptable salt thereof.

In some embodiments, the JAK inhibitor is a compound of Table 1, or a pharmaceutically acceptable salt thereof. The compounds in Table 1 are selective JAK1 inhibitors (selective over JAK2, JAK3, and TYK2).

TABLE 1
Examples of JAK inhibitors

| Comp. | | | |
|---|---|---|---|
| No. | Prep. | Name | Structure |
| 1 | US 2011/0224190 (Example 1) | {1-{1-[3-Fluoro-2-(trifluoro- methyl)isonicotinoyl]piperi- din-4-yl}-3-[4-(7H-pyrrolo [2,3-d]pyrimidin-4-yl)-1H- pyrazol-1-ylJazetidin-3-yl}- acetonitrile (itacitinib; also known as INCB039110) | 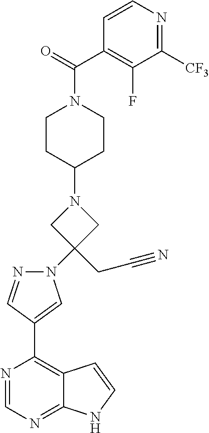 [SMILES: N#CCC1(n2cc(-c3ncnc4[nH]ccc34)cn2)CN(C2CCN(C(=O)c3ccnc(C(F)(F)F)c3F)CC2)C1] |
| 2 | US 2011/0224190 (Example 154) | 4-{3-(Cyanomethyl)-3-[4- (7H-pyrrolo[2,3-d]pyrimi- din-4-yl)-1H-pyrazol-1-yl] azetidin-1-yl}-N-[4-fluoro- 2-(trifluoromethyl)phenyl] piperidine-1-carboxamide | 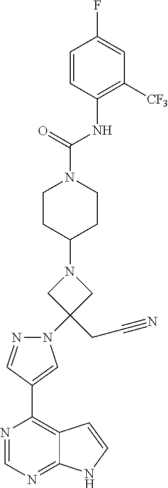 [SMILES: N#CCC1(n2cc(-c3ncnc4[nH]ccc34)cn2)CN(C2CCN(C(=O)Nc3ccc(F)cc3C(F)(F)F)CC2)C1] |
| 3 | US 2011/0224190 (Example 85) | [3-[4-(7H-pyrrolo[2,3-d] pyrimidin-4-yl)-1H-pyrazol- 1-yl]-1-(1-{[2-(trifluoro- methyl)pyrimidin-4-yl] carbonyl}piperidin-4-yl)- azetidin-3-yl]acetonitrile | 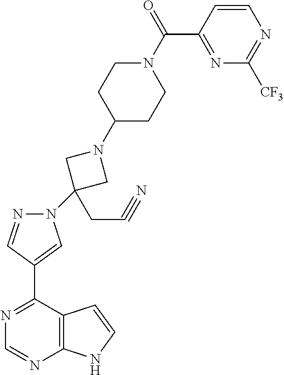 [SMILES: N#CCC1(n2cc(-c3ncnc4[nH]ccc34)cn2)CN(C2CCN(C(=O)c3ccnc(C(F)(F)F)n3)CC2)C1] |
| 4 | US 2014/0343030 (Example 7) | 4-[3-(cyanomethyl)-3-(3′,5′- dimethyl-1H, 1′H-4,4′- bipyrazol-1-yl)azetidin-1-yl] 2,5-difluoro-N-[(1S)-2,2,2- trifluoro-1-methylethyl] benzamide | 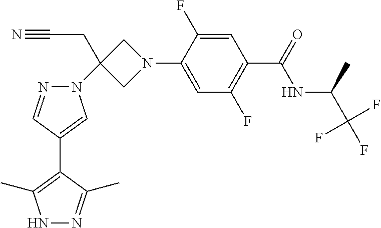 [SMILES: Cc1n[nH]c(C)c1-c1cnn(C2(CC#N)CN(c3cc(F)c(C(=O)N[C@@H](C)C(F)(F)F)cc3F)C2)c1] |
| 5 | US 2014/0121198 (Example 20) | ((2R,5S)-5-{2-[(1R)-1- hydroxy-ethyl]-1H-imidazo [4,5-d]thieno[3,2-b]pyridin- 1-yl}tetrahydro-2H-pyran- 2-yl)acetonitrile | 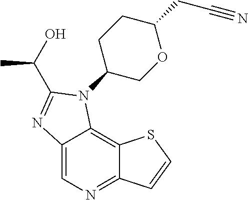 [SMILES: C[C@@H](O)c1nc2cnc3ccsc3c2n1[C@H]1CC[C@H](CC#N)OC1] |
| 6 | US 2010/0298334 (Example 2) | 3-[1-(6-chloropyridin-2-yl)- pyrrolidin-3-y1]-3-[4-(7H- pyrrolo[2,3-d]pyrimidin-4- yl)-1H-pyrazol-1-yl]propane- nitrile | 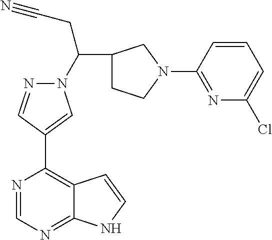 [SMILES: N#CCC(C1CCN(c2cccc(Cl)n2)C1)n1cc(-c2ncnc3[nH]ccc23)cn1] |
| 7 | US 2010/0298334 (Example 13c) | 3-(1-[1,3]oxazolo[5,4-b] pyridin-2-ylpyrrolidin-3- yl)-3-[4-(7H-pyrrolo[2,3-d] pyrimidin-4-y1)-1H-pyrazol- 1-yl]propanenitrile | 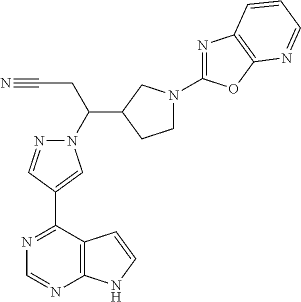 [SMILES: N#CCC(C1CCN(c2nc3cccnc3o2)C1)n1cc(-c2ncnc3[nH]ccc23)cn1] |
| 8 | US 2011/0059951 (Example 12) | 4-[(4-{3-cyano-2-[4-(7H- pyrrolo[2,3-d]pyrimidin-4- yl)-1H-pyrazol-1-yl]propyl}- piperazin-1-yl)carbonyl]-3- fluorobenzonitrile | 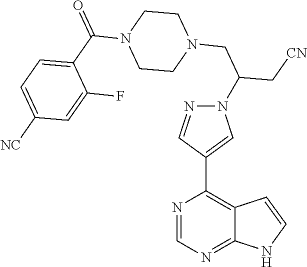 [SMILES: N#CCC(CN1CCN(C(=O)c2ccc(C#N)cc2F)CC1)n1cc(-c2ncnc3[nH]ccc23)cn1] |
| 9 | US 2011/0059951 (Example 13) | 4-[(4-{3-cyano-2-[3-(7H- pyrrolo[2,3-d]pyrimidin-4- yl)-1H-pyrrol-1-yl]propyl}- piperazin-1-yl)carbonyl]-3- fluorobenzonitrile | 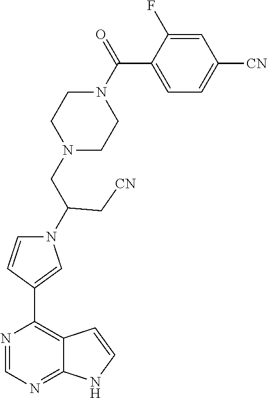 [SMILES: N#CCC(CN1CCN(C(=O)c2ccc(C#N)cc2F)CC1)n1ccc(-c2ncnc3[nH]ccc23)c1] |
| 10 | US 2012/0149681 (Example 7b) | [trans-1-[4-(7H-pyrrolo[2,3- d]pyrimidin-4-y1)-1H- pyrazol-1-y1]-3-(4-{[2- (trifluoromethyl)pyrimidin- 4-yl]carbonyl}piperazin-1- yl)cyclobutyl]acetonitrile | 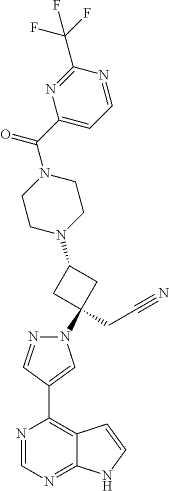 [SMILES: N#CC[C@]1(n2cc(-c3ncnc4[nH]ccc34)cn2)C[C@H](N2CCN(C(=O)c3ccnc(C(F)(F)F)n3)CC2)C1] |
| 11 | US 2012/0149681 (Example 157) | {trans-3-(4-{[4-[(3-hydroxy- azetidin-1-yl)methyl]-6- (trifluoromethyl)pyridin-2- yl]oxy}piperidin-1-yl)-1-[4- (7H-pyrrolo[2,3-d]pyrimi- din-4-yl)-1H-pyrazol-1-yl] cyclobutyl}acetonitrile | 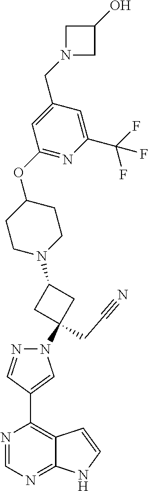 [SMILES: N#CC[C@]1(n2cc(-c3ncnc4[nH]ccc34)cn2)C[C@H](N2CCC(Oc3cc(CN4CC(O)C4)cc(C(F)(F)F)n3)CC2)C1] |
| 12 | US 2012/0149681 (Example 161) | {trans-3-(4-{[4-{[(2S)-2- (hydroxymethyl)pyrrolidin- 1-yl]methyl}-6-(trifluoro- methyl)pyridin-2-yl]oxy} piperidin-1-yl)-1-[4-(7H- pyrrolo[2,3-d]pyrimidin-4- yl)-1H-pyrazol-1-yl]cyclo- butyl}acetonitrile | 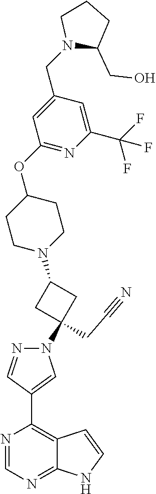 [SMILES: N#CC[C@]1(n2cc(-c3ncnc4[nH]ccc34)cn2)C[C@H](N2CCC(Oc3cc(CN4CCC[C@H]4CO)cc(C(F)(F)F)n3)CC2)C1] |
| 13 | US 2012/0149681 (Example 162) | {trans-3-(4-{[4-{[(2R)-2- (hydroxymethyl)pyrrolidin- 1-yl]methyl}-6-(trifluoro- methyl)pyridin-2-yl]oxy}- piperidin-1-yl)-1-[4-(7H- pyrrolo[2,3-d]pyrimidin- 4-yl)-1H-pyrazol-1-yl] cyclobutyl}acetonitrile | 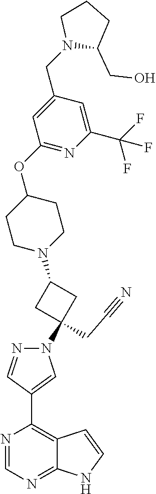 [SMILES: N#CC[C@]1(n2cc(-c3ncnc4[nH]ccc34)cn2)C[C@H](N2CCC(Oc3cc(CN4CCC[C@@H]4CO)cc(C(F)(F)F)n3)CC2)C1] |
| 14 | US 2012/0149682 (Example 20) | 4-(4-{3-[(dimethylamino)- methyl]-5-fluorophenoxy} piperidin-1-yl)-3-[4-(7H- pyrrolo[2,3-d]pyrimidin-4- yl)-1H-pyrazol-1-yl]butane- nitrile | 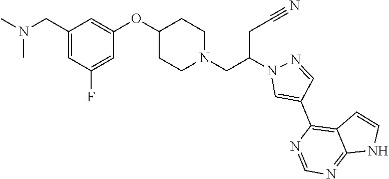 [SMILES: CN(C)Cc1cc(F)cc(OC2CCN(CC(CC#N)n3cc(-c4ncnc5[nH]ccc45)cn3)CC2)c1] |
| 15 | US 2013/0018034 (Example 18) | 5-{3-(cyanomethyl)-3-[4- (7H-pyrrolo[2,3-d]pyrimi- din-4-yl)-1H-pyrazol-1-yl] azetidin-1-y1}-N-isoprop- ylpyrazine-2-carboxamide | 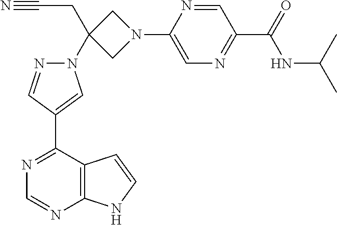 [SMILES: CC(C)NC(=O)c1cnc(N2CC(CC#N)(n3cc(-c4ncnc5[nH]ccc45)cn3)C2)cn1] |
| 16 | US 2013/0018034 (Example 28) | 4-{3-(cyanomethyl)-3-[4- (7H-pyrrolo[2,3-d]pyrimi- din-4-yl)-1H-pyrazol-1-yl] azetidin-1-yl}-2,5-difluoro- N-[(1S)-2,2,2-trifluoro-1- methylethyl]benzamide | 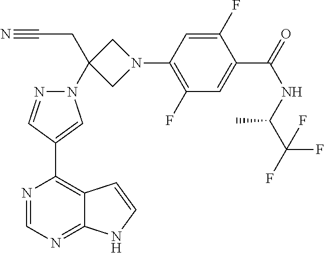 [SMILES: C[C@H](NC(=O)c1cc(F)c(N2CC(CC#N)(n3cc(-c4ncnc5[nH]ccc45)cn3)C2)cc1F)C(F)(F)F] |
| 17 | US 2013/0018034 (Example 34) | 5-{3-(cyanomethyl)-3-[4- (1H-pyrrolo[2,3-b]pyridin- 4-yl)-1H-pyrazol-1-yl]azeti- din-1-yl}-N-isopropylpyr- azine-2-carboxamide | 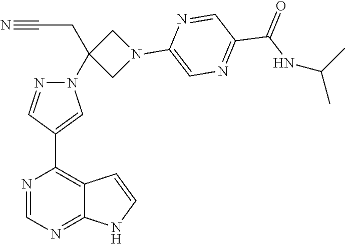 [SMILES: CC(C)NC(=O)c1cnc(N2CC(CC#N)(n3cc(-c4ncnc5[nH]ccc45)cn3)C2)cn1] |
| 18 | US 2013/0045963 (Example 45) | {1-(cis-4-{[6-(2-hydroxy- ethyl)-2-(trifluoromethyl)- pyrimidin-4-yl]oxycyclo- hexyl)-3-[4-(7H-pyrrolo [2,3-d]pyrimidin-4-yl)-1H- pyrazol-1-ylJazetidin-3-yl}- acetonitrile | 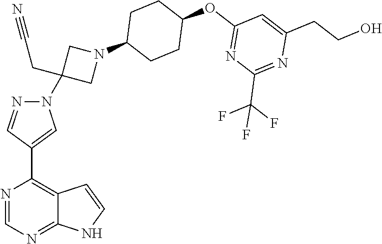 [SMILES: N#CCC1(n2cc(-c3ncnc4[nH]ccc34)cn2)CN([C@H]2CC[C@@H](Oc3cc(CCO)nc(C(F)(F)F)n3)CC2)C1] |
| 19 | US 2013/0045963 (Example 65) | {1-(cis-4-{[4-[(ethylamino)- methyl]-6-(trifluoromethyl)- pyridin-2-yl]oxy}cyclohex- yl)-3-[4-(7H-pyrrolo[2,3-d] pyrimidin-4-yl)-1H-pyrazol- 1-yl]azetidin-3-yl}acetonitrile | 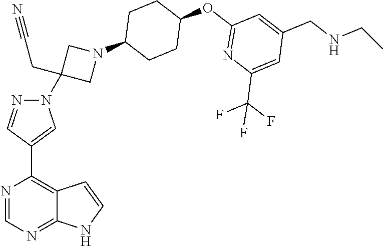 [SMILES: CCNCc1cc(O[C@H]2CC[C@@H](N3CC(CC#N)(n4cc(-c5ncnc6[nH]ccc56)cn4)C3)CC2)nc(C(F)(F)F)c1] |
| 20 | US 2013/0045963 (Example 69) | {1-(cis-4-{[4-(1-hydroxy- 1-methylethyl)-6-(trifluoro- methyl)pyridin-2-yl]oxy}- cyclohexyl)-3-[4-(7H-pyrrolo [2,3-d]pyrimidin-4-y1)-1H- pyrazol-1-ylJazetidin-3-yl}- acetonitrile | 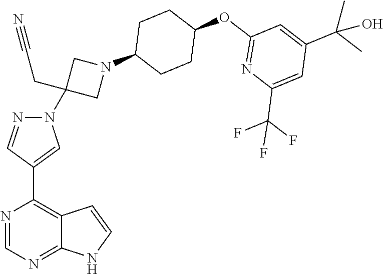 [SMILES: CC(C)(O)c1cc(O[C@H]2CC[C@@H](N3CC(CC#N)(n4cc(-c5ncnc6[nH]ccc56)cn4)C3)CC2)nc(C(F)(F)F)c1] |
| 21 | US 2013/0045963 (Example 95) | {1-(cis-4-{[4-{[(3R)-3- hydroxypyrrolidin-1-yl] methyl}-6-(trifluoromethyl)- pyridin-2-yl]oxy}cyclohex- yl)-3-[4-(7H-pyrrolo[2,3-d] pyrimidin-4-yl)-1H-pyrazol- 1-yl]azetidin-3-yl}acetonitrile | 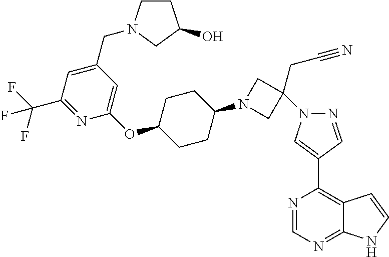 [SMILES: N#CCC1(n2cc(-c3ncnc4[nH]ccc34)cn2)CN([C@H]2CC[C@@H](Oc3cc(CN4CC[C@@H](O)C4)cc(C(F)(F)F)n3)CC2)C1] |
| 22 | US 2013/0045963 (Example 95) | {1-(cis-4-{[4-{[(3S)-3- hydroxypyrrolidin-1-yl] methyl}-6-(trifluoromethyl)- pyridin-2-yl]oxy}cyclohex- yl)-3-[4-(7H-pyrrolo[2,3-d] pyrimidin-4-yl)-1H-pyrazol- 1-yl]azetidin-3-yl}acetonitrile | 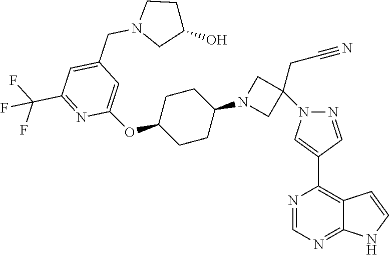 [SMILES: N#CCC1(n2cc(-c3ncnc4[nH]ccc34)cn2)CN([C@H]2CC[C@@H](Oc3cc(CN4CC[C@H](O)C4)cc(C(F)(F)F)n3)CC2)C1] |
| 23 | US 2014/0005166 (Example 1) | {trans-3-(4-{[4-({[(1S)-2- hydroxy-1-methylethyl] amino}methyl)-6-(trifluoro- methyl)pyridin-2-yl]oxy}- piperidin-1-yl)-1-[4-(7H- pyrrolo[2,3-d]pyrimidin-4- yl)-1H-pyrazol-1-yl]cyclo- butyl}acetonitrile | 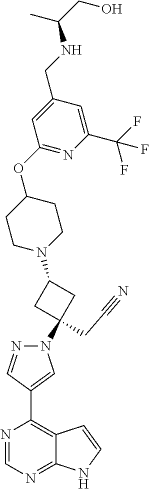 [SMILES: C[C@@H](CO)NCc1cc(OC2CCN([C@H]3C[C@](CC#N)(n4cc(-c5ncnc6[nH]ccc56)cn4)C3)CC2)nc(C(F)(F)F)c1] |
| 24 | US 2014/0005166 (Example 14) | {trans-3-(4-{[4-({[(2R)-2- hydroxypropyl]amino}meth- yl)-6-(trifluoromethyl)pyri- din-2-yl]oxy}piperidin-1- yl)-1-[4-(7H-pyrrolo[2,3-d] pyrimidin-4-yl)-1H-pyrazol- 1-yl]cyclobutyl}acetonitrile | 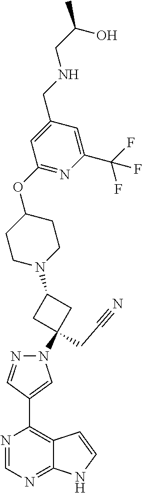 [SMILES: C[C@@H](O)CNCc1cc(OC2CCN([C@H]3C[C@](CC#N)(n4cc(-c5ncnc6[nH]ccc56)cn4)C3)CC2)nc(C(F)(F)F)c1] |
| 25 | US 2014/0005166 (Example 15) | {trans-3-(4-{[4-({[(2S)-2- hydroxypropyl]amino}meth- yl)-6-(trifluoromethyl)pyri- din-2-yl]oxy}piperidin-1- yl)-1-[4-(7H-pyrrolo[2,3-d] pyrimidin-4-yl)-1H-pyrazol- 1-yl]cyclobutyl}acetonitrile | 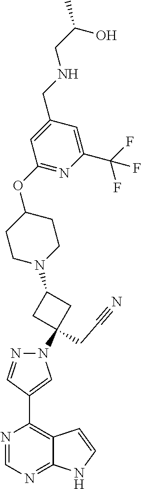 [SMILES: C[C@H](O)CNCc1cc(OC2CCN([C@H]3C[C@](CC#N)(n4cc(-c5ncnc6[nH]ccc56)cn4)C3)CC2)nc(C(F)(F)F)c1] |
| 26 | US 2014/0005166 (Example 20) | {trans-3-(4-{[4-(2-hydroxy- ethyl)-6-(trifluoromethyl)- pyridin-2-yl]oxy}piperidin- 1-yl)-1-[4-(7H-pyrrolo[2,3- d]pyrimidin-4-yl)-1H- pyrazol-1-yl]cyclobutyl}- acetonitrile | 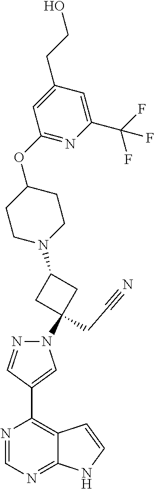 [SMILES: N#CC[C@]1(n2cc(-c3ncnc4[nH]ccc34)cn2)C[C@H](N2CCC(Oc3cc(CCO)cc(C(F)(F)F)n3)CC2)C1] |

In some embodiments, the JAK inhibitor is {1-{1-[3-fluoro-2-(trifluoromethyl) isonicotinoyl]piperidin-4-yl}-3 [4-(7H-pyrrolo[2,3-d]pyrimidin-4-yl)-1H-pyrazol-1-yl]azetidin-3-yl}acetonitrile, or a pharmaceutically acceptable salt thereof.

In some embodiments, the JAK inhibitor is {1-{1-[3-fluoro-2-(trifluoromethyl) isonicotinoyl]piperidin-4-yl}-3 [4-(7H-pyrrolo[2,3-d]pyrimidin-4-yl)-1H-pyrazol-1-yl]azetidin-3-yl}acetonitrile adipic acid salt.

The synthesis and preparation of {1-{1-[3-fluoro-2-(trifluoromethyl) isonicotinoyl]piperidin-4-yl}-3 [4-(7H-pyrrolo[2,3-d]pyrimidin-4-yl)-1H-pyrazol-1-yl]azetidin-3-yl}acetonitrile and the adipic acid salt of the same can be found, e.g., in US Patent Publ. No. 2011/0224190, filed Mar. 9, 2011, US Patent Publ. No. 2013/0060026, filed Sep. 6, 2012, and US Patent Publ. No. 2014/0256941, filed Mar. 5, 2014, each of which is incorporated herein by reference in its entirety.

In some embodiments, the JAK inhibitor is 4-[3-(cyanomethyl)-3-(3′,5′-dimethyl-1H, 1′H-4,4′-bipyrazol-1-yl) azetidin-1-yl]-2,5-difluoro-N-[(1S)-2,2,2-trifluoro-1-methylethyl]benzamide, or a pharmaceutically acceptable salt thereof.

In some embodiments, the JAK inhibitor is 4-[3-(cyanomethyl)-3-(3′,5′-dimethyl-1H,1′H-4,4′-bipyrazol-1-yl) azetidin-1-yl]-2,5-difluoro-N-[(1S)-2,2,2-trifluoro-1-methylethyl]benzamide phosphoric acid salt.

The synthesis and preparation of 4-[3-(cyanomethyl)-3-(3′,5′-dimethyl-1H,1′H-4,4′-bipyrazol-1-yl) azetidin-1-yl]-2,5-difluoro-N-[(1S)-2,2,2-trifluoro-1-methylethyl]benzamide and the phosphoric acid salt of the same can be found, e.g., in US Patent Publ. No. US 2014/0343030, filed May 16, 2014, which is incorporated herein by reference in its entirety.

In some embodiments, the JAK inhibitor is ((2R,5S)-5-{2-[(1R)-1-hydroxyethyl]-1H-imidazo[4,5-d]thieno[3,2-b]pyridin-1-yl}tetrahydro-2H-pyran-2-yl) acetonitrile, or a pharmaceutically acceptable salt thereof.

In some embodiments, the JAK inhibitor is ((2R,5S)-5-{2-[(1R)-1-hydroxyethyl]-1H-imidazo[4,5-d]thieno[3,2-b]pyridin-1-yl}tetrahydro-2H-pyran-2-yl) acetonitrile monohydrate.

Synthesis of ((2R,5S)-5-{2-[(1R)-1-hydroxyethyl]-1H-imidazo[4,5-d]thieno[3,2-b]pyridin-1-yl}tetrahydro-2H-pyran-2-yl) acetonitrile and characterization of the anhydrous and monohydrate forms of the same are described in US Patent Publ. No. 2014/0121198, filed Oct. 31, 2013 and US Patent Publ. No. 2015/0344497, filed Apr. 29, 2015, each of which is incorporated herein by reference in its entirety.

In some embodiments, the compounds of Table 1 are prepared by the synthetic procedures described in US Patent Publ. No. 2011/0224190, filed Mar. 9, 2011, US Patent Publ. No. 2014/0343030, filed May 16, 2014, US Patent Publ. No. 2014/0121198, filed Oct. 31, 2013, US Patent Publ. No. 2010/0298334, filed May 21, 2010, US Patent Publ. No. 2011/0059951, filed Aug. 31, 2010, US Patent Publ. No. 2012/0149681, filed Nov. 18, 2011, US Patent Publ. No. 2012/0149682, filed Nov. 18, 2011, US Patent Publ. 2013/0018034, filed Jun. 19, 2012, US Patent Publ. No. 2013/0045963, filed Aug. 17, 2012, and US Patent Publ. No. 2014/0005166, filed May 17, 2013, each of which is incorporated herein by reference in its entirety.

In some embodiments, JAK inhibitor is selected from the compounds, or pharmaceutically acceptable salts thereof, of US Patent Publ. No. 2011/0224190, filed Mar. 9, 2011, US Patent Publ. No. 2014/0343030, filed May 16, 2014, US Patent Publ. No. 2014/0121198, filed Oct. 31, 2013, US Patent Publ. No. 2010/0298334, filed May 21, 2010, US Patent Publ. No. 2011/0059951, filed Aug. 31, 2010, US Patent Publ. No. 2012/0149681, filed Nov. 18, 2011, US Patent Publ. No. 2012/0149682, filed Nov. 18, 2011, US Patent Publ. 2013/0018034, filed Jun. 19, 2012, US Patent Publ. No. 2013/0045963, filed Aug. 17, 2012, and US Patent Publ. No. 2014/0005166, filed May 17, 2013, each of which is incorporated herein by reference in its entirety.

### Pharmaceutical Formulations and Administration of a JAK Inhibitor

A JAK inhibitor can be administered in the form of a pharmaceutical composition. These compositions can be prepared in a manner well known in the pharmaceutical art, and can be administered by a variety of routes, depending upon whether local or systemic treatment is desired and upon the area to be treated. Administration may be oral, topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, a JAK inhibitor, in combination with one or more pharmaceutically acceptable carriers (excipients). In making the compositions of the disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10%, up to 20%, up to 30%, up to 40%, or up to 50% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

The compositions can be formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 1 and 11, e.g., from 1 to 8, from 3 to 8, from 5 to 9, or from 7 to 8. It will be understood that use of certain of the foregoing excipients, carriers, or stabilizers will result in the formation of pharmaceutical salts.

The therapeutically effective amount of a compound of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration (e.g., 0.1%, 0.5%, 1%, 2%, 3%, 4%, 5%, 6%, 7%, 8%, 9% or 10% w/v of the compound).

In some embodiments, a JAK inhibitor described herein (e.g., ruxolitinib) is administered at a dose of about 1 mg to 500 mg (e.g., a dose of about 1 mg, 5 mg, 10 mg, 15 mg, 20 mg, 25 mg, 30 mg, 40 mg, 50 mg, 75 mg, 100 mg, 150 mg, 200 mg, 250 mg, 300 mg, 350 mg, 400 mg, 450 mg, or 500 mg).

In some embodiments, a JAK inhibitor described herein (e.g., ruxolitinib) is administered orally at a dose of about 1 mg to 50 mg.

In some embodiments, a JAK inhibitor described herein (e.g., ruxolitinib) is administered orally twice per day at a dose of about 1 mg to 50 mg.

In some embodiments, a JAK inhibitor described herein (e.g., ruxolitinib) is administered orally at a dose of about 10 mg.

In some embodiments, a JAK inhibitor described herein (e.g., ruxolitinib) is administered orally twice per day at a dose of about 10 mg.

### Indications

An anti-CSF-1R antibody described herein (e.g., axatilimab) and a JAK inhibitor described herein (e.g., ruxolitinib) can be used in combination to treat chronic graft-versus-host disease (cGVHD). In some embodiments, the cGVHD is newly diagnosed cGVHD. In some embodiments, the cGVHD is moderate or severe cGVHD.

Another aspect comprises a combination of an anti-CSF-1R antibody described herein (e.g., axatilimab) and a JAK inhibitor described herein (e.g., ruxolitinib) for use in the treatment of cGVHD. In some embodiments, the cGVHD is newly diagnosed cGVHD. In some embodiments, the cGVHD is moderate or severe cGVHD.

Another aspect comprises a combination of anti-CSF-1R antibody described herein (e.g., axatilimab) and a JAK inhibitor described herein (e.g., ruxolitinib) in the manufacture of a medicament for treating cGVHD. In some embodiments, the cGVHD is newly diagnosed cGVHD. In some embodiments, the cGVHD is moderate or severe cGVHD.

In some embodiments, the JAK inhibitor is administered prior to administration of the anti-CSF-1R antibody.

In some embodiments, the JAK inhibitor is administered after the administration of the anti-CSF-1R antibody.

In some embodiments, the anti-anti-CSF-1R antibody and the JAK inhibitor are administered simultaneously or together.

The following are examples of the practice of the invention. They are not to be construed as limiting the scope of the invention in any way.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1: A Study of Axatilimab in Combination with Ruxolitinib in Subjects with Chronic Graft-Versus-Host Disease

This is a phase 2, open-label, randomized, parallel treatment group, multicenter study to evaluate the safety, tolerability, and preliminary activity of axatilimab in combination with ruxolitinib and to assess the contribution of axatilimab to the combination treatment effect in participants with newly diagnosed chronic graft-versus-host disease (cGVHD). Participants in the study have not previously received systemic treatment for moderate or severe cGVHD.

This study randomly assigns participants to one of the three treatment groups: axatilimab+ruxolitinib (Treatment Group A), ruxolitinib monotherapy (Treatment Group B), and corticosteroids (Treatment Group C). For patients randomized to steroid-free arms (A and B), rules for using steroids are pre-defined in the protocol; criteria for starting second-line therapy in patients randomized to the corticosteroids arm are also pre-defined. cGVHD response is assessed using objective measures delineated by standard 2014 NIH consensus criteria (Lee S J, Wolff D, Kitko C, et al. Measuring therapeutic response in chronic graft-versus-host disease. National Institutes of Health Consensus Development Project on Criteria for Clinical Trials in Chronic Graft-Versus-Host Disease: IV. The 2014 Response Criteria Working Group report. Biol Blood Marrow Transplant 2015; 21:984-999). This provides objective criteria for clinical decisions related to therapy changes.

Ruxolitinib monotherapy group (B) is included for an interpretation of the safety, efficacy and biomarkers data with the combination of axatilimab+ruxolitinib (A). The data from the ruxolitinib monotherapy arm enables evaluation of the contribution of axitilimab to the treatment effect of the combination.

Systemic corticosteroids represent the standard treatment in front-line cGVHD. Historical data with corticosteroids in front-line setting are limited and their value as reference data to compare with would be limited, given the heterogeneity of this patient population. Therefore, a corticosteroids treatment group (C) is included as a reference arm in this study to allow interpretation of the results from the two investigational, steroid-free treatment arms (A and B).

Participants are eligible to be included in the study only if all of the following inclusion criteria apply:
(1) ≥12 years of age at the time of informed consent;
(2) New onset moderate or severe cGVHD as defined by 2014 NIH Consensus Development Project Criteria, requiring systemic therapy (Jagasia et al., 2015, Biol Blood Marrow Transplant, 21 (3): 389-401, e381). Diagnosis of cGVHD requires at least 1 diagnostic feature of cGVHD or at least 1 distinctive feature plus additional tests such as biopsy, pulmonary function tests (PFTs), Schirmer test, or radiographic imaging showing cGVHD in the same or another organ. Participants with single-organ, genitourinary involvement or liver involvement as the only manifestation of cGVHD are not eligible;
(3) History of one allo-stem cell transplantation (any type of stem cell donor, any conditioning regimen and source of hematopoietic stem cells);
(4) Karnofsky Performance Scale (KPS) score ≥60% if aged 16 years and older; /Lansky Performance Scale (LPS) score ≥60% if younger than 16 years of age;
(5) Adequate hematologic function independent of platelet transfusion and growth factors for at least 7 days prior to study entry, i.e., absolute neutrophil count (ANC) ≥0.75×10⁹/L and platelet count ≥20×10⁹/L; and
(6) Willingness to avoid pregnancy or fathering children

Participants are excluded from the study if any of the following criteria apply:
(1) Patients who received more than one allo-stem cell transplantation (prior autologous hematopoietic cell transplantation is allowed);
(2) Patients with overlap cGVHD (i.e., the presence of features or characteristics of acute with simultaneous diagnostic and/or distinctive features of chronic GVHD). More specifically, participants with lower GI involvement (i.e., diarrhea) and/or cholestasis (i.e., total bilirubin >2 ULN) are not eligible;
Note: Prior history of aGVHD with resolution of symptoms is allowed.
(3) Patients who have received previous systemic treatment for cGVHD, including systemic corticosteroids and extracorporeal photopheresis (ECP). Participants who received topical or organ-specific therapies, as defined by the 2014 NIH guidelines for ancillary therapy and supportive care in clinical trials of cGVHD (Carpenter et al., 2015, Biol Blood Marrow Transplant, 21:1167-1187), started prior to Cycle 1 Day 1 are allowed and can be continued concomitantly with study treatment; Participants who received psoralen and ultraviolet A (PUVA) therapy or narrow-band ultraviolet B phototherapy for cGVHD are not eligible;
(4) Patients who received systemic corticosteroids within 2 weeks prior to Cycle 1 Day 1, regardless of indication. Participants with a history of aGVHD treated with systemic corticosteroids are eligible, provided corticosteroid taper was completed at least 2 weeks prior to Cycle 1 Day 1;
(5) Patients who initiated systemic treatment with calcineurin inhibitors (CNIs) or mTOR inhibitors within 2 weeks prior to Cycle 1 Day 1. Patients who received ongoing, concomitant treatment with a CNI (e.g., tacrolimus or cyclosporine) or an mTOR inhibitor (e.g., sirolimus or everolimus) is allowed under the following conditions: (i) CNI or mTOR inhibitor had been given for GVHD prophylaxis OR (ii) CNI or mTOR inhibitor was restarted for the management of aGVHD in a participant with a history of aGVHD in which aGVHD symptoms have resolved prior to screening. Doses may be adjusted for trough levels, with no increase in target trough range in the 2 weeks prior to start of study treatment on Cycle 1 Day 1;
(6) Prior treatment with a JAK inhibitor within 8 weeks before randomization (participants who received a JAK inhibitor for acute GVHD are eligible only if they achieved complete response or partial response to JAK inhibitor treatment and did not discontinue due to toxicity);
(7) Evidence of relapse of the primary hematologic disease, or receipt of treatment for relapse after the allo-stem cell transplantation was performed, including donor lymphocyte infusions (DLIs) for the treatment of molecular relapse (participants who have received a scheduled DLI as part of their transplant procedure and not for management of malignancy relapse are eligible);
(8) Maintenance therapy for the primary hematologic disease started within 4 weeks before initiation of study treatment (Day 1) or plans to start maintenance therapy after Day 1;
(9) Participant is receiving ongoing treatment with fluconazole at daily doses higher than 200 mg;
(10) History of acute or chronic pancreatitis. Note: Participants who had a defined pancreatitis etiology (e.g., pancreatitis due to gallstones, trauma/injury or medications such as asparaginase) may be enrolled if pancreatitis resolved prior to study entry;
(11) History of thromboembolic events, such as deep venous thrombosis, pulmonary embolism, stroke, myocardial infarction) in the past 6 months prior to study entry;
(12) Participant has active symptomatic myositis;
(13) Known HIV sero-positive status (HIV testing is performed at screening for participants with unknown HIV status);
(14) Patients with suspected active or latent tuberculosis (as confirmed by a positive QuantiFERON test or other tuberculosis blood test);
(15) Severe renal impairment, i.e., estimated creatinine clearance <30 ml/min measured or calculated by Cockcroft Gault equation in adults and Schwartz formula in pediatric participants, or end stage renal disease on dialysis (patients with CrCl 30 to 59 mL/min on treatment with fluconazole are not eligible);
(16) Impaired liver function defined as total bilirubin >1.5×ULN and/or ALT and AST >3×ULN in patients with no evidence of liver cGVHD;
(17) Impairment of gastrointestinal (GI) function (unrelated to GVHD) or GI disease (unrelated to GVHD) that may significantly alter the absorption of oral ruxolitinib (e.g., ulcerative diseases, uncontrolled nausea, vomiting, malabsorption syndrome, or small bowel resection) attributable to GVHD;
(18) Active, uncontrolled systemic bacterial, fungal, parasitic, or viral infection (CMV, EBV, HHV-6, BK virus); infections are considered controlled if appropriate therapy has been instituted and, at the time of screening, there is no evidence of infection worsening, such as hemodynamic instability attributable to sepsis, new symptoms, worsening physical signs or radiographic findings attributable to infection;
(19) Active hepatitis B virus (HBV) or hepatitis C virus (HCV). Participants with pre-transplant positive total HBc antibody or positive HCV antibody must have negative viral load for HBV and HCV at screening. Participants with unknown viral testing results prior to transplant must have viral load results confirming no evidence of active viral disease at screening;
(20) Currently active significant cardiac disease, such as uncontrolled arrhythmias, uncontrolled hypertension, or Class 3 or 4 congestive heart failure as defined by New York Heart Association, or a history of myocardial infarction or unstable angina within 6 months prior to randomization;
(21) Pregnant or breastfeeding;
(22) Vaccinated with live, attenuated vaccines within 4 weeks prior to the first dose of study treatment or anticipated need for live, attenuated vaccines while on study treatment;
(23) Treatment with an investigational agent, procedure, or device within 30 days of randomization, or within 5 half-lives of the investigational product, whichever is longer;
(24) Known allergies, hypersensitivity, or intolerance to any of the study medications, excipients, or similar compounds;
(25) Inability or unlikeliness of the participant to comply with the dose schedule and study evaluations, in the opinion of the investigator; and
(26) Any concurrent severe and/or uncontrolled medical conditions which, in the opinion of the investigator, could compromise participation in the study, pose a significant risk to the participant, or interfere with study results.

### Axatilimab Regimen:

The dose of axatilimab is 0.3 mg/kg IV (intravenous) Q2W (every 2 weeks). Axatilimab is administered by IV infusion over approximately 30 minutes, at the dosing/schedule corresponding to the Treatment Group (see below) to which the patient belongs. Patients should be weighed within 3 days prior to dosing. If the patient experiences either a weight loss or gain >10% compared to the weight used for the last dose calculation, the amount of study intervention must be recalculated to ensure that the dose administered is 0.3 mg/kg (+10%). For weight change <10%, the decision to recalculate the axatilimab dose can be in accordance with institutional practice.

### Ruxolitinib Regimen:

The dose of ruxolitinib is 10 mg given orally twice daily. Ruxolitinib should be taken at the same time each day, approximately 12 hours apart (morning and night) without regards to food.

### Systemic Corticosteroids Regimen:

The dose of systemic corticosteroids (e.g., methylprednisolone, prednisone, prednisolone) is 1.0 mg/kg/day prednisone equivalent. The methylprednisolone dose is converted to prednisone by multiplying the methylprednisolone dose by 1.25. Prednisolone may also be used at the same dose as prednisone.

### Justification for Axatilimab Dose

The dose of axatilimab is 0.3 mg/kg administered intravenously Q2W, which dosing regimen provided greater clinical benefit than 1 mg/kg Q2W and 3 mg/kg Q4W (every four weeks) when these regimens were evaluated in a clinical study investigating treatment with axatilimab given as a single agent to subjects with cGVHD after at least two prior lines of systemic therapy. The clinical benefits of the 0.3 mg/kg Q2W dose of axatilimab include:
(1) improved efficacy in the first 6 cycles, e.g., the primary endpoint of objective response rate (ORR) for the 0.3 mg/kg Q2W, 1 mg/kg Q2W, and 3 mg/kg Q4W doses were 73.8%, 66.7%, and 50%, respectively;
(2) reduction in modified Lee Symptom Scale (mLSS) score ≤7 points in 55% of participants in the 0.3 mg/kg Q2W cohort in the first 6 cycles; and
(3) improved safety profile, e.g., treatment-emergent adverse events (TEAEs) occurred in 6% of participants with 0.3 mg/kg Q2W, 22% with 1 mg/kg Q2W, and 18% with 3 mg/kg Q4W. The most common TEAEs were elevated aspartate aminotransferase/alanine aminotransferase (AST/ALT), creatine phosphokinase (CPK), lipase, amylase, and lactate dehydrogenase levels as well as periorbital edema, consistent with axatilimab's mechanism of action. The frequency of treatment-related AEs, any grade and ≥Grade 3, showed dose dependency, with lowest incidence at 0.3 mg/kg Q2W. Most infections were mild, with only 2 reported CMV infections in the higher dose cohorts; no CMV infections were reported with 0.3 mg/kg Q2W.

Additionally, post-hoc pharmacokinetic (PK) exposures, generated using a population PK/pharmacodynamic (PD) model developed using PK data from four axatilimab clinical studies demonstrated that the PK exposures in the five participants aged 12 to 17 years enrolled in cGVHD clinical studies were comparable to those observed in adult participants from the same dose groups. In addition, the developed population PK/PD model for axatilimab was extrapolated to simulate PK in pediatric patients aged 12 to 17 years, using standard allometric exponents of 0.75 for clearance-related parameters and 1.0 for volume of distribution-related parameters. The simulation results showed that the median predicted steady-state axatilimab exposures (AUC and Cₘₐₓ) in virtual pediatric patients aged 12 to 17 years at a 0.3 mg/kg Q2W dose were within the interquartile range of exposures in virtual adult patients at the same dose.

### Justification for Ruxolitinib Dose

The dose of ruxolitinib is 10 mg given orally twice daily, which is the approved starting dose of ruxolitinib for cGVHD after failure of 1 or 2 lines of systemic therapy.

### Treatment Groups:

Participants with newly diagnosed moderate or severe cGVHD are randomized to one of the three treatment groups. Enrollment is randomized in a 1:1:1 ratio to 1 of 3 treatment groups.
A: ruxolitinib 10 mg orally twice daily+axatilimab 0.3 mg/kg IV Q2W
B: ruxolitinib 10 mg orally twice daily
C: corticosteroids alone (starting dose 1 mg/kg/day prednisone equivalent)

### Changes to Dosing Schedule for Axatilimab

Participants randomized to axatilimab plus ruxolitinib treatment (Treatment Group A) who have had their Cycle 7 assessment and have achieved a partial response or complete response that has been sustained for at least 12 weeks may change their dose schedule from 0.3 mg/kg IV Q2W to 0.6 mg/kg IV Q4W. If, following a change in schedule from Q2W to Q4W, the participant experiences worsening of cGVHD or drug-related adverse events leading to axatilimab dosing delay, they may return to a Q2W schedule.

### Guidelines for New Systemic (2^{nd} Line) Therapy

Initiation of new systemic therapy should be considered in the following clinical circumstances:
Steroid-refractory or resistant cGVHD, defined per 2014 NIH consensus criteria (Lee S J, Wolff D, Kitko C, et al. Measuring therapeutic response in chronic graft-versus-host disease. National Institutes of Health Consensus Development Project on Criteria for Clinical Trials in Chronic Graft-Versus-Host Disease: IV. The 2014 Response Criteria Working Group report. Biol Blood Marrow Transplant 2015; 21:984-999 and Flowers M E D, Martin P J. How we treat chronic graft-versus-host disease. Blood 2015; 125:606-615) as follows:
   cGVHD manifestations progress despite the use of ≥1 mg/kg/day prednisone equivalent for at least 1 week; or
   cGVHD manifestations persist without improvement despite treatment with ≥0.5 mg/kg/day (or 1 mg/kg every other day) prednisone equivalent for at least 4 weeks;
Steroid-dependent cGVHD defined as inability to taper prednisone below 0.25 mg/kg/day (or >0.5 mg/kg every other day) in at least two unsuccessful attempts separated by at least 2 weeks;
Emergence of unacceptable toxicity due to the use of steroids.

### Objectives and Endpoints:

The primary objective of the study is to determine the preliminary efficacy of axatilimab in combination with ruxolitinib and to assess the contribution of axatilimab to the combination treatment effect in participants with cGVHD. The primary objective is evaluated by measuring objective response (OR) at 6 months, defined for each treatment group as complete response (CR) or partial response (PR) at 6 months (C7D1) in the absence of new systemic therapy for cGVHD. Response assessment will be based on the 2014 NIH Consensus Development Project on Criteria for Clinical Trials in cGVHD.

A secondary objective of the study is to determine the safety and tolerability of axatilimab in combination with ruxolitinib in participants with cGVHD. The secondary objective is evaluated by measuring the frequency and severity adverse events of (including serious adverse events) and changes in clinical and laboratory assessments.

A further secondary objective of the study is to evaluate the clinical benefit of axatilimab in combination with ruxolitinib in participants with cGVHD. This further secondary objective is evaluated by measuring the following endpoints: (1) duration of response (DOR) (in responders only), defined as the time from the date of first response (PR or CR) to the date of progression of cGVHD from nadir in any organ, start of new systemic therapy for cGVHD, or death from any cause, whichever occurs first. An additional measure of response durability will consider DOR as the time from the date of first response to the date of new systemic therapy for cGVHD or death from any cause, whichever occurs first; (2) proportion of participants with ≥7-point improvement in mLSS total score; (3) Event-Free Survival (EFS), defined as the time from the date of randomization to the date of addition or initiation of new systemic therapy for cGVHD, death due to any cause, or Day 1 in the case of treatment failure (i.e., non-CR or non-PR by Month 6), whichever occurs first. An additional measure of EFS will consider EFS2 as the time from the date of randomization to the date of progression based on best prior organ status (nadir in score level), addition or initiation of new systemic therapy for cGVHD, death due to any cause, or Day 1 in the case of treatment failure (i.e., non-CR or non-PR by Month 6), whichever occurs first (4) best overall response in the first 6 months and on study, based on the 2014 NIH Consensus Development Project on Criteria for Clinical Trials in cGVHD; (5) OR at 12 months, defined as CR or PR at 12 months (C14D1) in the absence of new systemic therapy for cGVHD; (6) proportion of participants who remain corticosteroid free at 4 weeks, 8 weeks, and 6 months; (7) organ-specific response in the first 6 cycles and on study, based on the 2014 NIH Consensus Development Project on Criteria for Clinical Trials in cGVHD; and (8) failure free survival (FFS), defined as the time from the date of randomization to the date of initiation of a new cGVHD treatment, malignancy relapse, or death due to any cause.

A further secondary objective of the study is to assess the pharmacokinetic (PK) of axatilimab when given in combination with ruxolitinib. This further secondary objective is evaluated by measuring PK parameters for axatilimab and ruxolitinib including maximum observed plasma or serum concentration (Cₘₐₓ), time to maximum concentration (tₘₐₓ), minimum observed plasma or serum concentration over the dose interval (Cₘᵢₙ), area under the plasma or serum concentration-time curve from time=0 to the last measurable concentration at time=t (AUC₀₋ₜ), area under the single-dose plasma or serum concentration-time curve extrapolated to time of infinity (AUC_{0-∞}), total systemic clearance (CL), volume of distribution during the elimination phase (V_{z}), and apparent terminal-phase disposition half-life (t_{1/2}) as deemed appropriate.

The exploratory objectives of the study are (1) to assess overall survival (OS) in participants with cGVHD, (2) to assess non-relapse mortality (NRM) in participants with cGVHD, (3) to assess cumulative corticosteroid exposure in participants with cGVHD, (4) to compare changes in health-related quality of life in participants with cGVHD, (5) to assess the immunogenicity of axatilimab in combination with ruxolitinib in participants with cGVHD, (6) to evaluate the effect of axatilimab in combination with ruxolitinib on bone turnover markers in participants with cGVHD, (7) to assess the pharmacodynamic (PD) profile of axatilimab in combination with ruxolitinib in participants with cGVHD, and (8) to evaluate the effect of axatilimab in combination with ruxolitinib on blood biomarkers in participants with cGVHD. The exploratory objectives are evaluated by measuring the following endpoints: (1) OS, defined as the time from the date of randomization to the date of death due to any cause, (2) NRM, defined as the time from the date of randomization to the date of death not preceded by underlying disease relapse/recurrence, (3) cumulative corticosteroid exposure during the first 3 and 6 months and time to corticosteroid initiation in the corticosteroid-alone treatment group, (4) changes in health-related quality of life as assessed by changes in symptom scores using EQ-5D-5L; (5) occurrence of specific antidrug antibodies to axatilimab, (6) changes in soluble markers for bone resorption and formation, including BAP and CTX, (7) change from baseline in CSF-1 and IL-34 levels and the association with cGVHD response, change from baseline in circulating monocyte phenotype (CD14/CD16), and frequency of immune cells in peripheral circulation, including T cells and B cells, and (8) changes from baseline and in response to treatment in circulating protein biomarkers, including inflammatory biomarkers.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating chronic graft-versus-host disease (cGVHD) in a human subject in need thereof, the method comprising administering to the human subject a therapeutically effective amount of a JAK inhibitor and an antibody that binds to colony stimulating factor 1 receptor (CSF-1R), wherein the antibody comprises a variable heavy (VH) domain comprising VH complementarity determining region (CDR) 1 (VHCDR1), VH CDR2, and VH CDR3, wherein:
the VH CDR1 comprises the amino acid sequence GFSLTTYGMGVG (SEQ ID NO:6);
the VH CDR2 comprises the amino acid sequence NIWWDDDKYYNPSLKN (SEQ ID NO:7); and
the VH CDR3 comprises the amino acid sequence IGPIKYPTAPYRYFDF (SEQ ID NO:8); and
wherein the antibody comprises a variable light (VL) domain comprising VL CDR1, VL CDR2, and VL CDR3, wherein:
the VL CDR1 comprises the amino acid sequence LASEDIYDNLA (SEQ ID NO:9);
the VL CDR2 comprises the amino acid sequence YASSLQD (SEQ ID NO:10); and
the VL CDR3 comprises the amino acid sequence LQDSEYPWT (SEQ ID NO:11).

2. The method of claim 1, wherein the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5).

3. The method of claim 1, wherein the antibody comprises a heavy chain and a light chain, and wherein the heavy chain comprises the amino acid sequence set forth in SEQ ID NO: 12 and the light chain comprises the amino acid sequence set forth in SEQ ID NO:3.

4. The method of claim 1, wherein the JAK inhibitor is ruxolitinib.

5. The method of claim 1, wherein the JAK inhibitor is itacitinib.

6. The method of claim 1, wherein the JAK inhibitor is 4-[3-(cyanomethyl)-3-(3′,5′-dimethyl-1H,1′HI-4,4′-bipyrazol-1-yl) azetidin-1-yl]-2,5-difluoro-N-[(1S)-2,2,2-trifluoro-1-methylethyl]benzamide or a pharmaceutically acceptable salt thereof, or ((2R,5S)-5-{2-[(1R)-1-hydroxyethyl]-1H-imidazo[4,5-d]thieno[3,2-b]pyridin-1-yl}tetrahydro-2H-pyran-2-yl) acetonitrile or a pharmaceutically acceptable salt thereof.

7. The method of claim 1, wherein the JAK inhibitor is a compound i of Formula I: 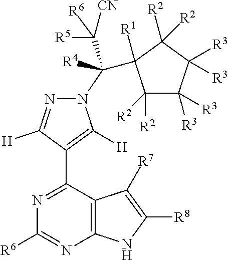
or a pharmaceutically acceptable salt thereof, wherein:
R¹ is selected from H and D;
each R² is independently selected from H and D, provided that each R² attached to a common carbon is the same;
each R³ is independently selected from H and D, provided that each R³ attached to a common carbon is the same;
R⁴ is selected from H and D;
each R⁵ is the same and is selected from H and D; and
R⁶, R⁷, and R⁸ are each independently selected from H and D; provided that when R¹ is H, each R² and each R³ are H, R⁴ is H, and each of R⁶, R⁷, and R⁸ is H, then each R⁵ is D.

8. The method of claim 1, wherein the cGVHD is newly diagnosed cGVHD.

9. The method of claim 1, wherein the cGVHD is moderate or severe cGVHD.

10. The method of claim 1, wherein the antibody is administered intravenously.

11. The method of claim 1, wherein the JAK inhibitor is administered orally.

12. The method of claim 1, wherein the JAK inhibitor is ruxolitinib and is administered orally at a dose of 1 mg to 50 mg.

13. (canceled)

14. The method of claim 1, wherein the JAK inhibitor is ruxolitinib and is administered orally at a dose of 10 mg.

15. (canceled)

16. The method of claim 1, wherein the antibody comprises a heavy chain and a light chain, wherein the heavy chain comprises the amino acid sequence set forth in SEQ ID NO: 12 and the light chain comprises the amino acid sequence set forth in SEQ ID NO:3, and wherein the antibody is administered intravenously at a dose of 0.3 mg/kg.

17. The method of claim 1, wherein the antibody comprises a heavy chain and a light chain, wherein the heavy chain comprises the amino acid sequence set forth in SEQ ID NO: 12 and the light chain comprises the amino acid sequence set forth in SEQ ID NO:3, and wherein the antibody is administered intravenously once every two weeks at a dose of 0.3 mg/kg.

18. The method of claim 1, wherein the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5), and wherein the JAK inhibitor is ruxolitinib.

19. The method of claim 1, wherein the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5), wherein the antibody is administered intravenously, and wherein the JAK inhibitor is ruxolitinib and is administered orally.

20. The method of claim 1, wherein the antibody comprises a heavy chain and a light chain, wherein the heavy chain comprises the amino acid sequence set forth in SEQ ID NO: 12 and the light chain comprises the amino acid sequence set forth in SEQ ID NO:3, and wherein the JAK inhibitor is ruxolitinib.

21. The method of claim 1, wherein the antibody comprises a heavy chain and a light chain, wherein the heavy chain comprises the amino acid sequence set forth in SEQ ID NO: 12 and the light chain comprises the amino acid sequence set forth in SEQ ID NO:3, wherein the antibody is administered intravenously, and wherein the JAK inhibitor is ruxolitinib and is administered orally.

22. The method of claim 1, wherein the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5), wherein the antibody is administered intravenously at a dose of 0.3 mg/kg, and wherein the JAK inhibitor is ruxolitinib and is administered orally at a dose of 10 mg.

23. The method of claim 1, wherein the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5), wherein the antibody is administered intravenously once every two weeks at a dose of 0.3 mg/kg, and wherein the JAK inhibitor is ruxolitinib and is administered orally twice per day at a dose of 10 mg.

24. The method of claim 1, wherein the antibody comprises a heavy chain and a light chain, wherein the heavy chain comprises the amino acid sequence set forth in SEQ ID NO: 12 and the light chain comprises the amino acid sequence set forth in SEQ ID NO:3, wherein the antibody is administered intravenously at a dose of 0.3 mg/kg, and wherein the JAK inhibitor is ruxolitinib and is administered orally at a dose of 10 mg.

25. The method of claim 1, wherein the antibody comprises a heavy chain and a light chain, wherein the heavy chain comprises the amino acid sequence set forth in SEQ ID NO: 12 and the light chain comprises the amino acid sequence set forth in SEQ ID NO:3, wherein the antibody is administered intravenously once every two weeks at a dose of 0.3 mg/kg, and wherein the JAK inhibitor is ruxolitinib and is administered orally twice per day at a dose of 10 mg.

26. The method of claim 1, wherein the antibody comprises a heavy chain and a light chain, wherein the heavy chain comprises the amino acid sequence set forth in SEQ ID NO:12 and the light chain comprises the amino acid sequence set forth in SEQ ID NO:3, and wherein the antibody is administered intravenously at a dose of 0.6 mg/kg.

27. The method of claim 1, wherein the antibody comprises a heavy chain and a light chain, wherein the heavy chain comprises the amino acid sequence set forth in SEQ ID NO:12 and the light chain comprises the amino acid sequence set forth in SEQ ID NO:3, and wherein the antibody is administered intravenously once every four weeks at a dose of 0.6 mg/kg.

28. The method of claim 1, wherein the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5), wherein the antibody is administered intravenously at a dose of 0.6 mg/kg, and wherein the JAK inhibitor is ruxolitinib and is administered orally at a dose of 10 mg.

29. The method of claim 1, wherein the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5), wherein the antibody is administered intravenously once every four weeks at a dose of 0.6 mg/kg, and wherein the JAK inhibitor is ruxolitinib and is administered orally twice per day at a dose of 10 mg.

30. The method of claim 1, wherein the antibody comprises a heavy chain and a light chain, wherein the heavy chain comprises the amino acid sequence set forth in SEQ ID NO: 12 and the light chain comprises the amino acid sequence set forth in SEQ ID NO:3, wherein the antibody is administered intravenously at a dose of 0.6 mg/kg, and wherein the JAK inhibitor is ruxolitinib and is administered orally at a dose of 10 mg.

31. The method of claim 1, wherein the antibody comprises a heavy chain and a light chain, wherein the heavy chain comprises the amino acid sequence set forth in SEQ ID NO: 12 and the light chain comprises the amino acid sequence set forth in SEQ ID NO:3, wherein the antibody is administered intravenously once every four weeks at a dose of 0.6 mg/kg, and wherein the JAK inhibitor is ruxolitinib and is administered orally twice per day at a dose of 10 mg.


SEQUENCE LISTING (12 sequences)

SEQ ID NO: 1 - protein, 972 aa, Homo sapiens
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 2 - protein, 453 aa, synthetic construct
EVTLKESGPALVKPTQTLTLTCTFSGFSLTTYGMGVGWIRQPPGKALEWLANIWWDDDKYYNPSLKNRLTISKDTSKNQVVLTMTNMDPVDTATYYCARIGPIKYPTAPYRYFDFWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - protein, 214 aa, synthetic construct
DIQMTQSPSSLSASVGDRVTITCLASEDIYDNLAWYQQKPGKAPKLLIYYASSLQDGVPSRFSGSGSGTDYTLTISSLQPEDFATYYCLQDSEYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 125 aa, synthetic construct
EVTLKESGPALVKPTQTLTLTCTFSGFSLTTYGMGVGWIRQPPGKALEWLANIWWDDDKYYNPSLKNRLTISKDTSKNQVVLTMTNMDPVDTATYYCARIGPIKYPTAPYRYFDFWGQGTMVTVS

SEQ ID NO: 5 - protein, 107 aa, synthetic construct
DIQMTQSPSSLSASVGDRVTITCLASEDIYDNLAWYQQKPGKAPKLLIYYASSLQDGVPSRFSGSGSGTDYTLTISSLQPEDFATYYCLQDSEYPWTFGGGTKVEIK

SEQ ID NO: 6 - protein, 12 aa, synthetic construct
GFSLTTYGMGVG

SEQ ID NO: 7 - protein, 16 aa, synthetic construct
NIWWDDDKYYNPSLKN

SEQ ID NO: 8 - protein, 16 aa, synthetic construct
IGPIKYPTAPYRYFDF

SEQ ID NO: 9 - protein, 11 aa, synthetic construct
LASEDIYDNLA

SEQ ID NO: 10 - protein, 7 aa, synthetic construct
YASSLQD

SEQ ID NO: 11 - protein, 9 aa, synthetic construct
LQDSEYPWT

SEQ ID NO: 12 - protein, 452 aa, synthetic construct
EVTLKESGPALVKPTQTLTLTCTFSGFSLTTYGMGVGWIRQPPGKALEWLANIWWDDDKYYNPSLKNRLTISKDTSKNQVVLTMTNMDPVDTATYYCARIGPIKYPTAPYRYFDFWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG